(19) United States
(12) Patent Application Publication
(10) Pub. No.: US 2004/0064053 A1
(43) Pub. Date: Apr. 1, 2004

(54) Diagnostic fluorescence and reflectance

(76) Inventors: Sung K. Chang, Austin, TX (US); Yvette Mirabal, Houston, TX (US); Michele Follen, Houston, TX (US); Anais Malpica, Houston, TX (US); Urs Utzinger, Tucson, AZ (US); Gregg Staerkel, Pearland, TX (US); Dennis Cox, Houston, TX (US); E. Neely Atkinson, Houston, TX (US); Calum MacAulay, Vancouver (CA); Rebecca Richards-Kortum, Austin, TX (US)

Correspondence Address: FULBRIGHT & JAWORSKI L.L.P., 600 CONGRESS AVE., SUITE 2400, AUSTIN, TX, 78701, US

(21) Appl. No.: 10/262,610

(22) Filed: Sep. 30, 2002

(51) Int. Cl. A61B 6/00

(52) U.S. Cl. 600/478

(57) ABSTRACT

Systems and methods are described for improved diagnostic fluorescence and reflectance. A method of detecting tissue abnormality in a tissue sample in vivo detects a set of reflectance spectra emitted from a tissue sample as a result of illumination with an excitation light from a fiber optic probe that has at least one collection fiber positioned at a source-detector separation, and determining if the tissue sample is normal or abnormal based on the resulting reflectance spectra. Another method of detecting tissue abnormality in a tissue sample in vivo includes illuminating the tissue sample in vivo with at least one electromagnetic radiation wavelength selected to cause the tissue sample to produce a set of fluorescence intensity spectra indicative of tissue abnormality, detecting the resulting fluorescence intensity spectra, and determining if the tissue sample is normal or abnormal based on the resulting fluorescence intensity spectra. Yet another method of detecting tissue abnormality in a tissue sample in vivo includes combining the two methods described above.

### STATEMENT AS TO RIGHTS TO INVENTIONS MADE UNDER FEDERALLY-SPONSORED RESEARCH AND DEVELOPMENT

This invention was made with United States Government support under contract to UT-Austin awarded by the National Cancer Institute (PO1-CA82710). The Government may have certain rights in this invention.

### BACKGROUND OF THE INVENTION

1. Field of the Invention

The invention relates generally to the field of biological imaging. More particularly, the invention relates to the detection of tissue abnormalities such as, but not limited to, cervical cancer. Specifically, a preferred implementation of the invention relates to the detection of cervical cancer through use of fluorescence spectroscopy, reflectance spectroscopy, or a combination of fluorescence and reflectance spectroscopy.

2. Discussion of the Related Art

Papanicoloau smear, in which a small sample of cells collected from the cervical epithelium are diagnosed under the microscope by an expert, is at present the most comprehensive means of screening and detecting cervical cancer. Although the Papanicoloau smear has been effective in reducing the mortality due to cervical cancer, it is highly dependent on the skill of the investigator. In fact, the mean sensitivity and specificity in screening using Papanicoloau smear are 73% and 63%, respectively.

Fluorescence spectroscopy has been investigated as an effective and non-invasive method for screening and detecting cervical cancer. Fluorescence spectroscopy of the tissue is affected by various optical interactions. Changes in index of refraction in the tissue and scatterers such as the cell nuclei causes scattering of light. Hemoglobin molecules are significant light absorbers at certain wavelengths. Light is also absorbed by chromophores, which then emit fluorescent light. Biological chromophores such as NADH and flavins are closely related to cellular metabolism. Scattering, absorption and fluorescence properties convey significant morphologic, cytologic and histo-pathologic information of the tissue under investigation.

A number of clinical trials have shown that fluorescence spectroscopy has promise for in vivo, real time detection of cervical neoplasia. Typically in these trials, fluorescence emission spectra are measured at one to three excitation wavelengths and diagnostic algorithms are developed retrospectively based on features of these spectra. One study reports a sensitivity and specificity of 92% and 90% using one excitation wavelength at 337 nm and those of 82% and 68%, respectively, when 3 excitation wavelengths at 337 nm, 380 nm and 460 nm were used. At least one group has reported a sensitivity and specificity of 93% and 94%, respectively, at 337 nm excitation. Based on the fluorescence spectroscopy algorithm developed by Ramanujam et al. (Photochem. Photobiol. 1996), researchers have developed a system to image fluorescence from cervical epithelium at multiple excitation emission wavelength pairs. Over 100 patients have been evaluated with this device; the initial data from the study show that the device discriminates between precancerous cervical cancer lesion from normal tissue with a sensitivity and specificity of 98% and 95.4%. Recently, a similar device, which incorporates the ability to measure both reflectance and fluorescence was used to measure the colposcopically visible cervical epithelium. 136 patients were measured in the colposcopy setting, of which 111 patients were included for analysis. An algorithm was derived to recognize cervices with CIN 2 or greater. Encouraging sensitivities and specificities were reported (97% and 70% respectively). However in both studies, algorithm results are reported from the same data set used to derive the algorithm; thus, estimates of sensitivity and specificity may be high due to over-training bias.

An important limitation of past studies is that the selection of excitation wavelength was based either on availability of a source or on the basis of small, in vitro studies surveying many different excitation wavelengths. It is well known that the optical properties of epithelial tissue differ in vitro, implying that different excitation wavelengths may be optimal for in vivo studies.

Recently, several groups have developed spectroscopic systems which enable measurement of fluorescence emission spectra at many excitation wavelengths in vivo. These emission spectra can be assembled into an excitation emission matrix (EEM), which contains the fluorescence intensity as a function of both excitation and emission wavelength. These systems provide a convenient way to characterize the autofluorescence properties of epithelial tissue over the entire UV-visible spectrum. While these research level systems enable clinical trials to determine the optimal excitation wavelengths for diagnostic purposes, they are not suited for office-based diagnosis. Cost-effective devices, using a smaller number of optimized excitation wavelengths will be required to allow the technology to enter wide scale clinical practice.

In view of shortcomings in the art, it would be advantageous to carry out in vivo measurements of fluorescence EEMs and analyze these data to determine the optimal excitation wavelengths for diagnosis of cervical neoplasia, and to estimate the sensitivity and specificity at this combination of excitation wavelengths. It would also be advantageous to determine the optimal source-detector separation combinations for the diagnosis of cervical neoplasia in reflectance measurements, and the optimal combination of the reflectance source-detector separations and fluorescence excitation wavelengths.

### SUMMARY OF THE INVENTION

There is a need for the following embodiments. Of course, the invention is not limited to these embodiments.

According to an aspect of the invention, a method comprises: providing a tissue sample, sequentially illuminating the tissue sample in vivo with an excitation light from a fiber optic probe; detecting the set of reflectance spectra emitted from the tissue sample as a result of illumination with the excitation light from the fiber optic probe with the fiber optic probe; and determining from the set of reflectance intensity spectra whether the tissue sample is normal or abnormal. The fiber optic probe comprises an at least one collection fiber positioned at an at least one source-detector separation selected from the group consisting of 250 μm separation, 1.1 mm separation, 2.1 mm separation, and 3.0 mm separation.

According to another aspect of the invention, a method of detecting tissue abnormality in a tissue sample in vivo comprises providing a tissue sample; sequentially illuminating the tissue sample in vivo with a first and second electromagnetic wavelength; detecting the set of fluorescence intensity spectra emitted from the tissue sample as a result of illumination with each of the wavelengths; and determining from the set of fluorescence intensity spectra whether the tissue sample is normal or abnormal. The first electromagnetic wavelength is selected from the range of 330-350 nm and the second electromagnetic wavelength is selected from the range of 370-450 nm. According to other aspects of the invention, the first electromagnetic wavelength is selected from the range of 330-340 nm and the second electromagnetic wavelength is selected from the range of 410-420 nm, the first electromagnetic wavelength is selected from the range of 330-350 nm and the second electromagnetic wavelength is selected from the range of 400-450 nm, or a single electromagnetic radiation wavelength is selected from the range 370-400 nm, or three electromagnetic radiation wavelengths are selected from the ranges of 330-340 nm, 350-380 nm, and 400-450 nm.

Yet another method of detecting tissue abnormality in a tissue sample in vivo comprises: providing a tissue sample; sequentially illuminating the tissue sample in vivo with an excitation light and a first, second, and third electromagnetic radiation wavelength from a fiber optic probe; detecting the set of fluorescence intensity spectra emitted from the tissue sample as a result of illumination with each of the wavelengths; detecting the set of reflectance spectra emitted from the tissue sample as a result of illumination with the excitation light from the fiber optic probe with the fiber optic probe; and determining from the set of fluorescence intensity spectra and/or the reflectance spectra whether the tissue sample is normal or abnormal. The fiber optic probe comprises an at least one collection fiber positioned at an at least one source-detector separation selected from the group consisting of 250 μm separation, 1.1 mm separation, 2.1 mm separation, and 3.0 mm separation. The first electromagnetic wavelength is selected from the range 330-360 nm, the second electromagnetic wavelength is selected from the range 420-430 nm, and the third electromagnetic wavelength is selected from the range 460-470 nm. According to other aspects of the invention, the first electromagnetic wavelength is selected from the range 350-360 nm, the second electromagnetic wavelength is selected from the range 420-430 nm, and the third electromagnetic wavelength is 460 nm. According to another aspect of the invention, two wavelengths are used. The first electromagnetic wavelength is selected from the range 330-360 nm and the second electromagnetic wavelength is selected from the range 460 nm; or the first electromagnetic wavelength is selected from the range 330-360 nm and the second electromagnetic wavelength is 470 nm; or the first electromagnetic wavelength is selected from the range 330-350 nm and the second electromagnetic wavelength is 470-480 nm. According to another aspect of the invention, the first electromagnetic wavelength is selected from the range 330-350 nm, the second electromagnetic wavelength is selected from the range 460-470 nm, and two source detection separations are selected from the group consisting of 250 μm separation, 1.1 mm separation, 2.1 mm separation, and 3.0 mm separation.

### BRIEF DESCRIPTION OF THE DRAWINGS

FIG. 1 illustrates a three emission average spectra of pre and post menopausal women (a) SN sites from the screening setting, (b) SN sites from the referral setting, and (c) SIL sites from the referral setting for (left to right) 337 nm, 380 nm and 460 m excitation. Error bars represent one standard deviation.

FIG. 2 illustrates an average spectra of Caucasian and African-American women from (a) SN sites and (b) SIL sites in the referral setting. Error bars represent plus and minus one standard deviation.

FIG. 3 illustrates a fluorescence intensity from squamous epithelial cells of the cervix at 340 nm excitation, 440 nm emission daily throughout the cycle in ten patients. The plots are the mean fluorescence intensity of the three sites measured each day for each patient, and the error bars represent the maximum and the minimum values of the three sites. Blue, green and red dots represent menstrual, proliferative and secretory phases of the cycle, respectively. Note that data is plotted against the day of the cycle, which is not necessarily the same as the measurement day.

FIG. 4 illustrates a mean sensitivity and specificity for all of the single excitation wavelengths, and the top 25 performing combinations of two, three and four excitation wavelengths for distinguishing pairwise combinations of histopathologic categories at ESL of 75%. Dark gray and light gray plots are sensitivity and specificity, respectively. Error bars show plus and minus one standard deviation. SN=Squamous Normal, CN=Columnar Normal, LG=Low-Grade Squamous Intraepithelial Lesion, HG=High grade Squamous Intraepithelial Lesion

FIG. 5 illustrates a histogram indicating the frequency of occurrence of each excitation wavelength in the top 25 performing combinations of 2 excitation wavelengths. ESL was 75%. Results are shown for pairwise discrimination between SN and HGSIL.

FIG. 6 illustrates an average reflectance spectra by tissue type for different source-detector separations. Tissue spectra have been normalized by spectra from a standard solution containing 1.05 μm diameter polystyrene microspheres.

FIG. 7 illustrates a sensitivity and specificity obtained from a single source-detector separation, and the top performing combinations of two, three and four source detector separations for distinguishing pairwise combinations of histopathologic categories at ESL of 65%. Blue and maroon bars indicate sensitivity and specificity, respectively. SN=Squamous Normal, CN=Columnar Normal, LG=Low-Grade Squamous Intraepithelial Lesion, HG=High grade Squamous Intraepithelial Lesion

FIG. 8 illustrates a sensitivity and specificity obtained from the combinations of one, two, three and four reflectance and fluorescence spectra for distinguishing pairwise combinations of histopathologic categories at ESL of 65%. Blue and maroon bars indicate sensitivity and specificity, respectively. SN=Squamous Normal, CN=Columnar Normal, LG=Low-Grade Squamous Intraepithelial Lesion, HG=High grade Squamous Intraepithelial Lesion

FIG. 9A illustrates (left) a canonical score calculated from morphologic features assessed from Feulgen stained histopathologic tissue sections. FIG. 9B (right) shows a canonical score calculated from architectural features assessed from Feulgen stained histopathologic tissue sections.

FIG. 10 illustrates a typical fluorescence EEM of (a) a normal squamous site, (b) a normal columnar site and (c) HGSIL from the same patient. Excitation wavelength is shown on the ordinate and emission wavelength is shown on the abscissa. Contour lines connect points of equal fluorescence intensity.

FIG. 11 illustrates a mean sensitivity and specificity for all the single excitation wavelength, and the top 25 performing combinations of two, three and four excitation wavelengths for distinguishing pairwise combinations of histopathologic categories at ESL of 75%. Dark gray and light gray plots are sensitivity and specificity, respectively. Error bars show plus and minus one standard deviation.

FIG. 12 illustrates a sensitivity and specificity for ensemble classifiers made up of the top 25 performing combinations of three and four excitation wavelengths at ESLs of 65%, 75%, 85% and 95%, x-axis label shows the different combinations. For example, ‘3SIG65’ denotes 3 wavelength combinations at 65% ESL. Results are shown for ensemble classifiers requiring agreement among 50% (dashed line), 80% (dotted line) and 100% (solid line) of the individual algorithms. Results are shown for algorithms which discriminate (a) SN against CN, (b) SN against HGSIL, (c) SN against CIN 2 and CIN 3, (d) CN against LGSIL, and (e) CN against HPV and CIN 1 emission wavelength is shown on the abscissa. Contour lines connect points of equal fluorescence intensity.

FIG. 13 shows a histogram indicating the frequency of occurrence of each excitation wavelength in the top 25 performing combinations of 2 excitation wavelengths. ESL was 75%. Results are shown for pairwise discrimination between (a) SN and CN, (b) SN and LGSIL, (c) SN and HGSIL, (d) CN and LGSIL, and (e) CN and HGSIL.

FIG. 14 shows a variable excitation light source assembly, a fiber-optic delivery and collection probe, and a spectral multi-channel analyzer/polychromator assembly.

FIG. 15 is a schematic diagram of the distal end of the probe: [A] fluorescence excitation (white circles in dark gray field) and collection (black circles on dark gray field) fiber bundle, [B] reflectance illumination fiber (white circle in white field) and [positions 0-3] reflectance collection fibers (black circles in white field).

FIG. 16 shows average reflectance spectra by tissue diagnostic classification for different source-detector separations: (a) position 0; (b) position 1; (c) position 2; (d) position 3. Error bars indicate +/−one standard deviation. Tissue spectra have been normalized by standard spectra from a suspension of 6.25% by volume polystyrene microspheres (1.02 μm diameter).

FIG. 17 shows the sensitivity and specificity for top-performing source-detector separation combinations when taken one, two, three, and four at a time for a given pairwise combination of histopathologic categories at an ESL=65%. Gray and black bars indicate sensitivity and specificity, respectively. SN=squamous normal, CN=columnar normal, LGSIL=low-grade squamous intraepithelial lesion, HGSIL=high-grade squamous intraepithelial lesion.

FIG. 18 shows the sensitivity and specificity for top-performing source-detector separation combinations when taken one, two, three, and four at a time for a given pairwise combination of histopathologic categories at an ESL=95%. Gray and black bars indicate sensitivity and specificity, respectively. SN=squamous normal, CN=columnar normal, LGSIL=low-grade squamous intraepithelial lesion, HGSIL=high-grade squamous intraepithelial lesion.

FIG. 19 illustrates typical in vivo spectra for cervical tissue: (a) normal squamous; (b) normal columnar, and (c) carcinoma in situ, measured with the system. In the left column, reflectance spectra at four different source-detector separations, normalized by a standard microsphere solution, are shown. In the right column, fluorescence excitation-emission matrix data are shown.

FIG. 20A illustrates the average of the top 10 values of sensitivity (black) and specificity (gray) for a diagnostic differentiation pair, when the classification features of the four source-detector separations and the 16 excitation wavelengths are combined one, two, or three at a time for an ESL=65%. Dots indicate the sensitivity/specificity of the best performing combination. FIG. 20B illustrates the average of the top 10 values of sensitivity (black) and specificity (gray) for a diagnostic differentiation pair, when the classification features of the four source-detector separations and the 16 excitation wavelengths are combined one, two, or three at a time for an ESL=95%. Dots indicate the sensitivity/specificity of the best performing combination.

FIG. 21 illustrates the average sensitivity (black) and specificity (gray) of the five best performing classification combinations, for each pair-wise comparison of diagnostic categories (ESL=65%). Results are shown for reflectance and fluorescence spectra combined (R+F) when selecting combinations of up to three features at a time, fluorescence spectra alone (F) when selecting combinations of up to three excitation wavelengths at a time, and reflectance spectra alone (R) when selecting combinations of up to four source-detector separations at a time.

FIG. 22 shows the classification features that predominated, in terms of frequency of feature inclusion, in consideration of the ten best combinations of features when taken one, two, or three at a time for pair-wise discrimination between diagnostic categories. The most significant features are shown for the analyses of reflectance alone, fluorescence alone, and reflectance/fluorescence combined.

FIG. 23 illustrates the frequency histograms of classification features, the four reflectance source detector (s-d) separation positions and the 16 fluorescence excitation wavelengths (λₑₓ) when cumulatively considering the ten best combinations of features when taken one, two, or three at a time for pair-wise discrimination between diagnostic categories where: (a) SN vs. CN; (b) SN vs. LGSIL; (c) SN vs. HGSIL; (d) CN vs. LGSIL; and (e) CN vs. HGSIL. ESL=65%.

FIG. 24 illustrates the average spectra of correctly classified tissue measurements of a diagnostic class (heavy black line) and individual spectra of the misclassified tissue measurements within each diagnostic class (thin black lines), using the feature combination that gives the best discrimination performance, when only four features (330 nm, 360 nm, 430 nm, and 470 nm) are available, for a given diagnostic pairing, where: (a) SN vs. CN; (b) SN vs. LGSIL; (c) SN vs. HGSIL; (d) CN vs. LGSIL; and (e) CN vs. HGSIL.

### DETAILED DESCRIPTION

An analysis that examined whether fluorescence spectra could be correlated with biographical variables such as patient age, race or menopausal status was conducted. Results indicate that while a subset of these variables are important, data processing strategies can remove their effects. However, data from larger trials stratified by these variables may enable new data processing approaches that incorporate these biographical variables and lead to algorithms with higher sensitivity and specificity. The fluorescence spectra was examined for variations throughout the menstrual cycle. Results indicate that while there are small changes that occur throughout the cycle, these changes do not achieve statistical significance. Furthermore, diagnostic algorithms perform equally well when applied to data collected throughout the cycle. Thus, measurement of fluorescence can be done anytime during the cycle. It was determined that emission spectra from only two excitation wavelengths are required to achieve optimal diagnostic performance, and that sensitivity and specificity do not significantly increase as excitation wavelengths are added. This interim analysis yielded the excitation wavelengths needed to design a multispectral digital colposcope. It was also found that reflectance spectroscopy also may contain significant diagnostic information, and that the combination of fluorescence and reflectance spectroscopy provides the best discriminatory capability. Quantitative cytology and histology can be used discriminate HG lesions from other classes well. Tissue architecture changes are taken as the basis for the discriminative capability shown.

A series of clinical trials to develop and evaluate the performance of diagnostic and screening algorithms based on fluorescence and reflectance spectroscopy were conducted. It has been shown that diagnostic algorithms can classify tissue samples as diseased or non-diseased based on fluorescence emission collected from the intact cervix. Such algorithms can discriminate normal tissue from squamous intraepithelial lesions (SILs) and low grade SILs from high grade SILs with a similar sensitivity and significantly improved specificity relative to colposcopy in expert hands. However, it was noted in early clinical studies that there is great variability in the fluorescence spectra collected from different patients, even within a single histo-pathological category. For example, peak fluorescence intensities of normal tissues can vary by more than a factor of five from patient to patient, but within a single patient the standard deviation is usually less than 25% of the average value. Because of this large variation between patients, early data analysis was performed in a paired manner. However, paired analysis required that a normal and abnormal site be measured for each patient. Subsequent data analysis methods removed the need for paired data, but current diagnostic algorithms still require normalization and mean-scaling of the data as part of the preprocessing to reduce the effects of the interpatient variations.

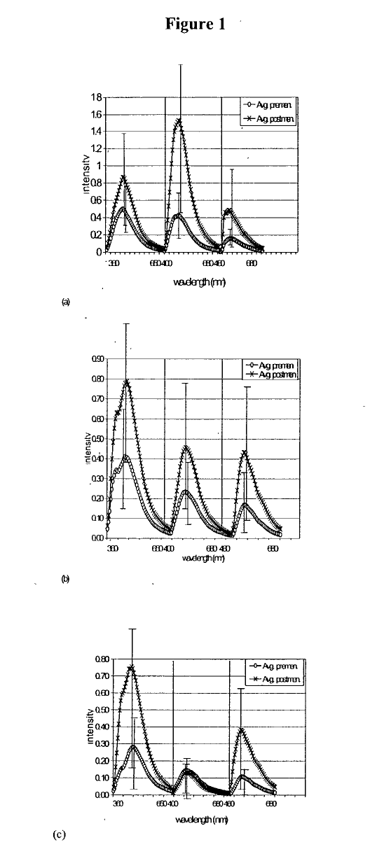

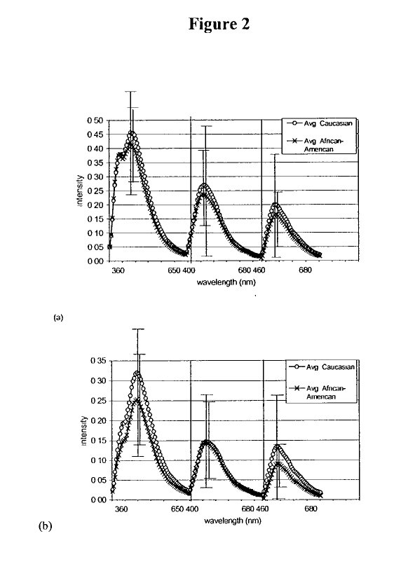

An analysis to examine the effects of biographical variables on tissue fluorescence spectra was carried out. The analysis was performed using data collected from two previously published clinical trials; one study measured spectra from 395 sites in 95 patients referred to a colposcopy clinic with abnormal Pap smears, and the second study measured spectra from 204 sites in 54 patients self-referred for screening and expected to have a normal Pap smear. A diagnostic algorithm was developed and has been described in detail. For this analysis, data about age, race, menstrual cycle, menstrual status (pre-, peri-, post-), and smoking were collected. Principal component analysis on normalized and non-normalized data was compared. An ANOVA was performed based on age, menopausal status, race and smoking. There are clear intensity differences observed with menopausal status; post-menopausal patients exhibit higher emission intensities (FIG. 1). This difference is not due entirely to age; age appears to also influence intensity with higher emission intensities seen with greater age. Caucasian women had slightly increased emission intensities compared to African-Americans (FIG. 2). Current and former smokers had slightly increased emission intensities compared to non-smokers. Differences associated with biographical variables may be tested in larger studies, which stratify adequately for these variables. The addition of these biographical variables in the pre-processing of data could improve algorithm performance and applicability.

The results described herein demonstrate that statistically significant differences in the principal components which describe spectral data can arise due to factors such as the patient's age, or menopausal status, even when the pathological diagnosis of the measured tissue is the same. This suggests a clear biological basis for the spectral variations seen clinically among women of various ages. The increased fluorescence intensity with increasing age and menopausal status is consistent with a large proportion of the signal originating in the stroma, which contains collagen and elastin. Older, post-menopausal women are known to experience a thinning of the cervical epithelium, and may experience changes in collagen cross-linking as well. Further statistical work may be necessary in a larger data set to gain a complete understanding of the particular effects of biographical variables on fluorescence spectra. In particular, a greater number of measurements may be needed from older, postmenopausal women. Clinical trials are ongoing in which participants are stratified based on their age and hormonal status to ensure that the data is from a group that is well distributed across these variables. This may permit development and training of an algorithm that can account for such inter-patient variations. Current preprocessing techniques, normalization and mean-scaling, are limiting because normalization ignores intensity differences between spectra and mean-scaling requires an equal number of normal and abnormal sites per patient. Alternative preprocessing methods, which could account for the differences due to patient age or menopausal status without normalization, for example, may offer improvements to algorithm performance and applicability.

Menstrual Cycle

Fluorescence spectroscopy for the screening and diagnosis of cervical pre-cancer has demonstrated promising results. Clinical trials show a sensitivity and specificity of 86% and 74% in the diagnostic colposcopy clinic and 75% and 80% in the screening setting. In both diagnostic and screening settings, peak fluorescence intensity varies by more than an order of magnitude from one patient to another. In an attempt to further improve the sensitivity and specificity of real-time diagnosis, it is necessary to understand and control for the sources of this inter-patient variability. In general, the intra-patient variation is less than the inter-patient variation. However, the biological basis for these variations is not well understood. Previous work has shown that race and smoking do not account for these variations, while age and menopausal status may play a role.

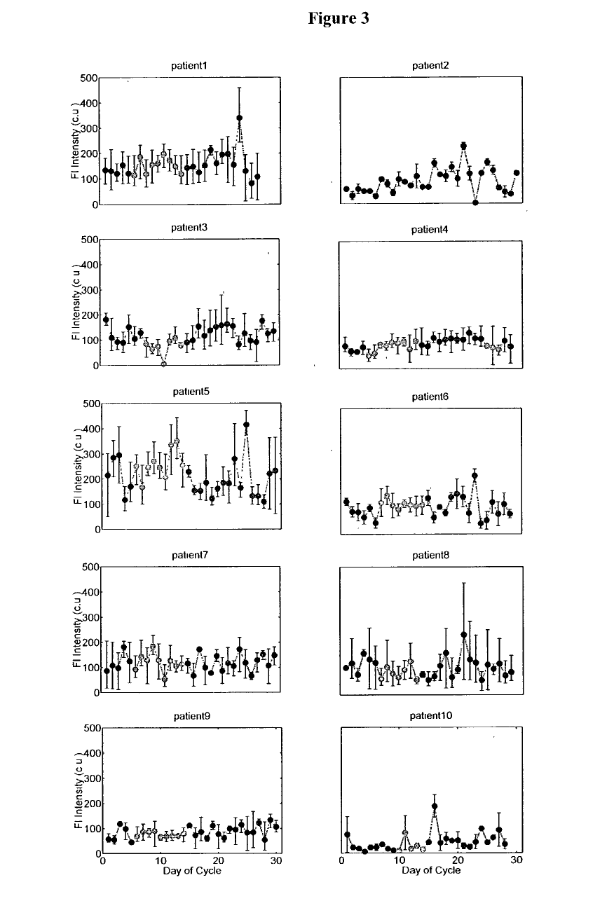

The inventors have assessed one possible cause of inter-patient variation in fluorescence spectroscopy of the cervix: the menstrual cycle. Ten patients with no history of an abnormal Pap smear were seen daily throughout on average 30 consecutive days of their cycle. Fluorescence excitation-emission matrices were measured from three cervical sites on each patient. FIG. 3 shows the fluorescence intensity versus day in cycle averaged over three sites. Principal Component Analysis was used to determine which spectral regions varied with the day of the cycle. Classification was performed to assess the influence of menstrual cycle on pre-cancer diagnosis. Variations in the principal component scores and the redox ratio values show that the fluorescence emission spectra at 340-380 nm excitation appear to correlate with the cell metabolism of the cervical epithelium throughout the menstrual cycle; these changes do not achieve statistical significance however. There are insufficient data points from biopsy proven SIL to calculate a reliable sensitivity. Thus, the menstrual cycle affects intra-patient variation but does not appear to cause significant level of inter-patient variation. It therefore doesn't need to be controlled for in optical detection strategies based on fluorescence spectroscopy, because the algorithm performs well irrespective of day in cycle.

Interim Analysis of Diagnostic Fluorescence Trial and Reflectance Fluorescence Combined Trial

An important limitation of past studies is that the selection of excitation wavelength(s) was based either on the availability of a source or on the basis of small, in vitro studies surveying many different excitation wavelengths. These emission spectra can be assembled into an excitation emission matrix (EEM), which contains the fluorescence intensity as a function of both excitation and emission wavelength. These systems provide a convenient way to characterize the autofluorescence properties of epithelial tissue over the entire UV-visible spectrum. One aim of this disclosure was to analyze these data to determine the optimal excitation wavelengths for diagnosis of cervical neoplasia, and to estimate the sensitivity and specificity at this combination of excitation wavelengths.

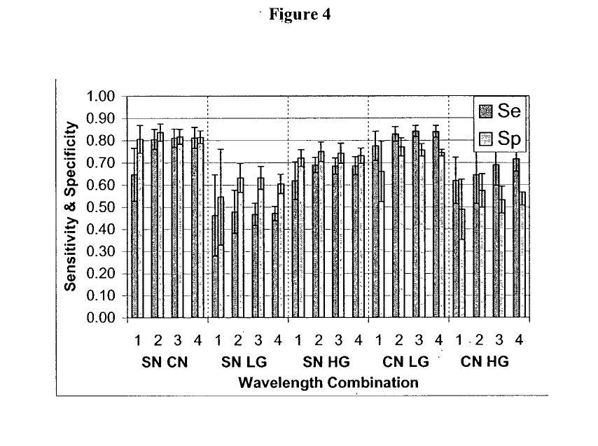

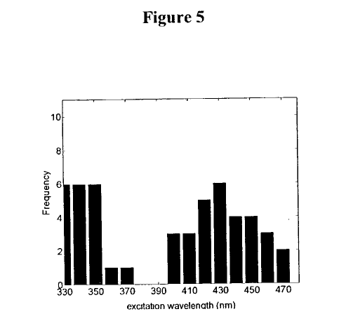

Fluorescence excitation-emission matrices (EEMs) were measured in vivo from 351 sites in the first 146 patients of an earlier diagnostic fluorescence and reflectance spectroscopy study with biospy. Data were analyzed to determine which combination of excitation wavelengths yields diagnostic algorithms with the greatest sensitivity and specificity. FIG. 4 illustrates the effect of increasing the number of excitation wavelengths for algorithms that classify the important histopathological categories in the cervix. FIG. 4 shows the mean and the standard deviation of the cross-validated sensitivities and specificities from the top-25 excitation wavelength combinations for each pair of classes tested at an eigenvector significance level (ESL) of 75%. In general, the performance of the algorithms developed for a pair of classes improves when the number of excitation wavelengths used in the combination is increased from one to two; however, adding a third or a fourth excitation wavelength does not generally improve diagnostic performance. The histopathologic categories of interest were squamous normal areas (SN), columnar normal areas (CN), areas of low-grade squamous intraepithelial lesions (LG), and high-grade squamous intraepithelial lesions (HG). The algorithm performs well for separating SN vs. CN, SN vs. HGSIL, and CN vs. LGSIL. In contrast, lower performance is observed for separating SN vs. LGSIL and CN vs. HGSIL.

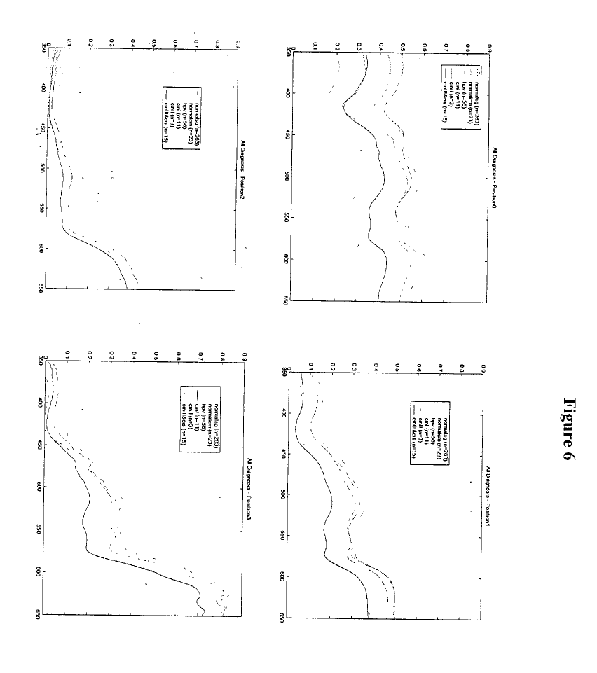

In order to select the wavelengths most promising for discrimination between histopathologic classes of cervical tissue, those excitation wavelengths that resulted in highest diagnostic performance were explored. Histograms were created that indicated the frequency of occurrence of each excitation wavelength in the top 25 performing combinations of two excitation wavelengths at an ESL of 75% for all pair-wise discriminations. Results are shown in FIG. 5. FIG. 5 shows the histogram for discrimination between SN and HGSIL, and indicates that 330-350 nm occurs most frequently, and wavelengths ranging from 420-450 nm also occur frequently. It was found that 330-340 nm, 360-400 nm and 410-460 nm excitation may yield the best performance for discriminating among all pairs of diagnostic categories. In this study it was found that the proportion of samples that are HGSIL may influence performance. Furthermore stratification of samples within LGSIL (HPV, CIN 1) and HGSIL (CIN 2, CIN 3) also appears to influence diagnostic performance.

In other words, fluorescence EEMs of SN, CN, LGSIL and HGSIL show characteristic differences that can be used to discriminate among histopathologic classes. It was found that, to discriminate between pairs of histologic classifications, performance increases when data from two excitation wavelengths are combined. However, adding data from a third or a fourth excitation wavelength does not generally increase diagnostic performance. Best discrimination was found between spectra from SN and CN, between SN and HGSIL and between CN and LGSIL, with sensitivity and specificity averaging 70-80%.

Using similar analysis methods, the diagnostic potential of reflectance spectroscopy in an interim analysis of data from the large diagnostic trial of fluorescence and reflectance spectroscopy with biopsy was examined. FIG. 6 shows average reflectance spectra at four different source-detector separations (position 0: 250 μm separation, position 1: 1.1 mm separation, position 2: 2.1 mm separation, and position 3: 3.0 mm separation). Spectra show valleys due to hemoglobin absorption at 420 nm, 542 and 577 nm. Clear differences are seen in the average spectra at all positions, with reflectance of squamous normal tissues being highest, columnar normal tissues being lowest, and a gradual decrease in reflectance from CIN 1-3.

TABLE 1
Best Source-Detector Separations

| Diagnostic Classes | Se | Sp | Separations |
|---|---|---|---|
| SN vs. CN | 0.89 | 0.79 | 0, 1 |
| SN vs. LG | 0.69 | 0.55 | 3 |
| SN vs. HG | 0.81 | 0.73 | 1 |
| CN vs. LG | 0.76 | 0.89 | 0, 1, 3 |
| CN vs. HG | 0.75 | 0.89 | 0, 1, 2 |

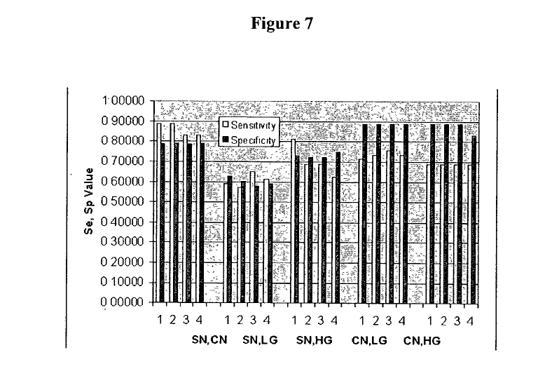

FIG. 7 shows the sensitivity and specificity for the best performing combination of 1, 2, 3 and 4 source detector separations. The diagnostic performance is high when using only a single source detector separation, and does not noticeably increase when data from additional source detector separations is included. Table 1 gives the sensitivity and specificity for the combination of source-detector separations which yielded the highest sum of the sensitivity and specificity by cross-validation.

The sensitivity and specificity exceed that achieved with fluorescence alone for distinguishing most categories. Best results are obtained for discriminating squamous normal vs columnar normal, columnar normal tissue vs High Grade SILs and for columnar normal vs. low grade SILs.

Similar analyses to examine the combination of fluorescence and reflectance spectroscopy were carried out. Algorithms based on the Mahalanobis distance were used to evaluate combinations of up to three reflectance features from amongst the four source detector separations and fluorescence features from amongst sixteen excitation wavelengths. Best results were found using fluorescence spectra that had been normalized to a maximum value of unity.

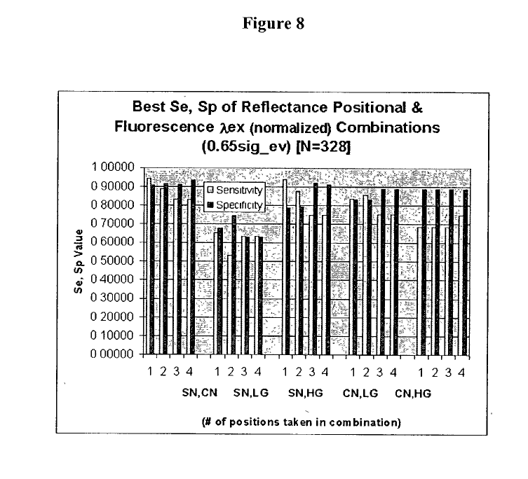

FIG. 8 shows the sensitivity and specificity for the best performing combination of 1, 2, 3 and 4 reflectance and fluorescence features. The diagnostic performance is high when using only a single source detector separation, and does not noticeable increase when data from additional source detector separations is included. The combination of fluorescence and reflectance spectroscopy provides improved performance for discrimination of all categories and indicates the importance of completing large trials evaluating both of these technologies in combination.

Table 2 gives the sensitivity and specificity for the combination of reflectance source-detector separations and fluorescence excitation wavelengths which yielded the highest sum of the sensitivity and specificity by cross-validation. Note that best results are achieved using only fluorescence spectroscopy to discriminate squamous normal and columnar normal tissues, as well as to discriminate columnar normal and low grade SILs. Best results are obtained using reflectance alone to discriminate columnar normal tissues and high grade SILs, while both techniques are required to discriminate squamous normal tissues from low grade and high grade SILs.

TABLE 2
Best Combinations of Reflectance and Fluorescence Spectra

| Diagnostic Classes | Se | Sp | Spectra |
|---|---|---|---|
| SN vs. CN | 0.94 | 0.91 | Fluorescence: 340 nm |
| SN vs. LG | 0.75 | 0.61 | Reflectance: 1, 3 |
| | | | Fluorescence: 380, 430 nm |
| SN vs. HG | 0.94 | 0.80 | Fluorescence: 340 nm |
| | | | Reflectance: 2 |
| CN vs. LG | 0.94 | 0.83 | Fluorescence: 340, 380 nm |
| CN vs. HG | 0.94 | 0.78 | Reflectance: 0, 2, 3 |

In previous works, the highest sensitivity and specificity obtained using fluorescence spectroscopy alone at 340, 380 and 460 nm was 86% and 74% respectively. It is encouraging to note that these studies combining reflectance spectroscopy with fluorescence EEM measurements show increased sensitivity and specificity for all diagnostic class combinations except squamous normal vs. low grade SIL.

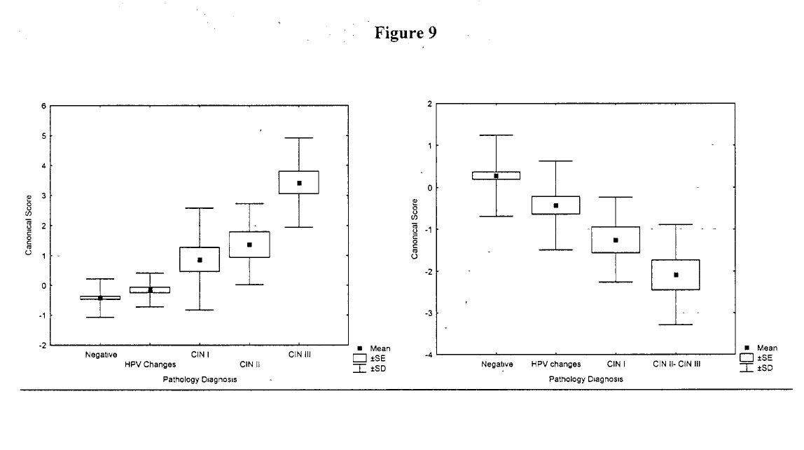

An interim analysis of the quantitative histopathologic data acquired in a large diagnostic trial with biopsy was also conducted. Quantitative images were obtained from Feulgen stained tissue sections that were mapped by the pathologist to the diagnostic areas in H&E stained serial sections. Morphologic features were quantified in each biopsy, and a discriminant analysis was run to compare the mean and standard deviation of each feature for histopathologically normal and abnormal biopsies. Six features were found to have statistically significant differences. These features were then used to derive a canonical score for each biopsy. FIG. 9A shows the mean, standard error and standard deviation of the canonical score for each diagnostic category in a box-whisker plot. Similarly, this analysis was repeated using architectural features assessed from the Feulgen stained sections. In this analysis, four features were selected and the corresponding canonical score was calculated and plotted against the pathology grade (FIG. 9B). There is a clear continuum of changes as lesion severity increases.

### EXAMPLES

Specific embodiments of the invention will now be further described by the following, nonlimiting examples which will serve to illustrate in some detail various features. The following examples are included to facilitate an understanding of ways in which the invention may be practiced.

### EXAMPLE 1

### Materials and Methods: Reflectance Only

Instrumentation

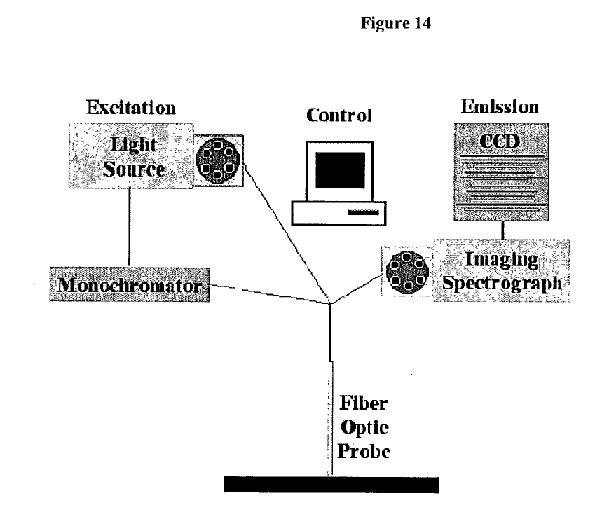

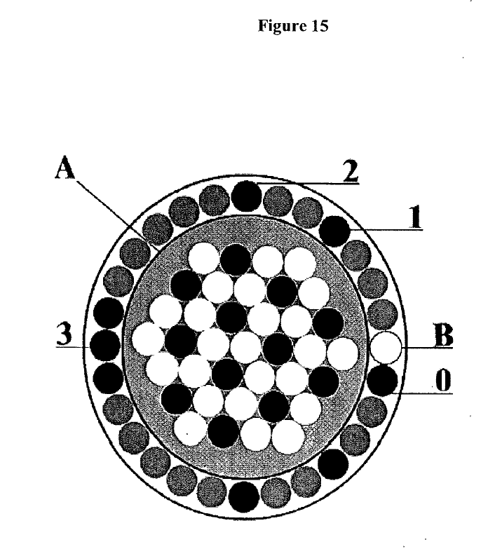

Briefly, the system consists of three main components: (1) a light source assembly that provides broadband excitation using a Xenon arc lamp; (2) a fiber-optic probe that directs excitation light to tissue and collects diffusely reflected light; and (3) an optical assembly with a polychromator. FIG. 14 illustrates the system. The probe, illustrated in FIG. 15, utilizes nine optical fibers placed in direct contact with the tissue [200 μm diameter, numerical aperture (NA)=0.2]. One fiber provides broadband illumination. Eight collection fibers at four different source-detector separations (position 0: 250 μm separation, position 1: 1.1 mm separation, position 2: 2.1 mm separation, position 3: 3.0 mm separation) collect diffusely reflected light.

Clinical Measurements

Following colposcopic examination, but prior to biopsy, a fiber optic probe was advanced through the speculum and placed in gentle contact with the cervix. Spectroscopic measurements were obtained from up to two colposcopically abnormal cervical sites, one colposcopically normal cervical site covered with squamous epithelium, and if visible, one colposcopically normal cervical site covered with columnar epithelium. Following spectroscopic measurements, all sites interrogated with the fiber-optic probe were biopsied.

Within two hours of each patient measurement, standard spectra were measured. As a positive control, reflectance spectra were measured from a one cm pathlength cuvette containing a suspension of 1.02 μm diameter polystyrene microspheres (6.25% by vol.), to mimic the optical properties of tissue. As a negative control, reflectance spectra were measured with the probe tip immersed in a large container of distilled water. With the negative control, very low levels of reflectance are expected due to the small index mismatch between glass and water. Low signal levels indicated acceptable levels of stray light, dark current, or other sources of background signal.

Biopsies were fixed and submitted for permanent section. Four-micron thick sections were stained with hematoxylin and eosin and Feulgen stained. All routinely stained cytology and pathology specimens were submitted for diagnosis by experienced cytologists and pathologists, who were blinded to the results of the spectroscopy. Two cytologists read each Pap smear, and discrepant cases were reviewed a third time for consensus diagnosis by the study cytopathologist. Also, two pathologists read each biopsy, with discrepant cases reviewed a third time for consensus diagnosis by the study histopathologist. Diagnostic classification categories included normal tissues, HPV infection, grade 1 cervical intra-epithelial neoplasia (CIN-1), grade 2 cervical intra-epithelial neoplasia (CIN 2), grade 3 cervical intra-epithelial neoplasia (CIN 3), and carcinoma in situ (CIS) using standard histopathologic criteria. Normal tissues were divided into two categories based on colposcopic impression: normal squamous epithelium (SN) and normal columnar epithelium (CN). LGSILs include HPV and CIN 1 and HGSILs include CIN 2, CIN 3, and CIS.

Data processing & Statistical Analysis

Three investigators blinded to the pathologic results reviewed all spectra. Spectra indicating evidence of user or instrument error, such as probe slippage, were discarded from further analysis. To remove the effects of the source spectrum, variations in the illumination intensity, and the wavelength-dependant response of the detection system, the tissue spectra was divided by the corresponding spectra measured from the microsphere standard. This normalization was performed at each source-detector separation. While this procedure results in spectra that describe the transport of light in tissue relative to that in microsphere suspension, the transport properties of microspheres are well known and can easily be described using Monte-Carlo based models.

Reflectance data from a single measurement site are represented as a matrix containing calibrated reflectance intensity as a function of source-detector separation position and emission wavelength. Spectra for each of the four positions were column vectors containing 121 intensity measurements corresponding to emission wavelengths from 355 nm to 655 nm in 2.5 nm increments.

Reflectance spectra were then analyzed to determine which source-detector separations contained the most diagnostically useful information to separate each of the different types of tissue found in the cervix. An algorithm to separate each pairwise combination of diagnostic categories was developed and evaluated relative to the gold standard of colposcopically directed biopsy. In comparing all pairs of diagnostic classes, it is possible to determine which categories differ spectroscopically and assess where these differences are greatest. This information can then be used to develop multi-step classification algorithms to determine the tissue type of an unknown sample based on its reflectance spectrum. Algorithm development consisted of the following steps: (1) selection of the source-detector separations to analyze; (2) data reduction using principal component analysis (PCA); (3) feature selection and classification using Mahalanobis distance with cross-validation. Each step is described in detail below.

To identify the optimal combination of source-detector separations, all possible combinations were evaluated when taken one, two, three, or four at a time. There were a total of 15 combinations considered-four of one, six of two, four of three, and one of four.

Prior to PCA, vectors containing the reflectance spectra for the source-detector separations of interest were concatenated into a single vector. A data matrix was assembled from these vectors, including all measurements from the two diagnostic categories being compared. Eigenvectors of the corresponding covariance matrix were then calculated; those accounting for up to 65, 75, 85, and 95% of the total variance were retained for algorithm development. The fraction of the total variance accounted for is denoted as the eigenvector significance level (ESL). For each eigenvector, a principal-component score was calculated for each sample in the data matrix.

Classification functions were then formed to assign a sample to one of the two given classes. Classification was based on the Mahalanobis distance, r2, the multivariate measure of the separation of a data point (x) from the mean (xm) of a dataset in n-dimensional space (Eq.

1). This distance is given as:

r²=(x−xₘ)′·Cₓ⁻¹·(x−xₘ) Eq. 1

where Cₓ is the covariance matrix. The multivariate distance between the measurement to be classified and the means of the two histopathologic categories were calculated, and the sample was assigned to the group it was closest to in multivariate space.

The performance of classification depends on the principal-component scores included for analysis. From the available pool of eigenvectors the single principal-component score yielding the best initial performance was identified, then the score that improved this performance most was selected. This process was repeated until performance was no longer enhanced by the addition of principal components, or until all components were selected. The sensitivity and specificity of diagnostic algorithms for each combination of source-detector separations were then evaluated relative to the histopathologic diagnosis. To reduce the risk of overtraining, cross-validation was used to estimate algorithm performance. In this process, a single sample from a whole data set is temporarily removed and a classification algorithm is developed using the remaining data. The algorithm is applied to the held-out data. Each sample in the dataset was held out in turn and the sensitivity and specificity were calculated by comparing the classification result of each sample to histopathologic diagnosis—diseased tissue was taken as the positive sample relative to either squamous or columnar normal tissue and columnar normal tissue was taken as the positive sample relative to squamous normal tissue. Overall diagnostic performance was evaluated as the sum of the sensitivity and specificity.

### EXAMPLE 2

### Results: Reflectance Only

Data Set

The data set consisted of spectra from 324 sites in 161 patients that were deemed adequate. Table 3 indicates the number of measurements within each diagnostic category. Tissues with acute or chronic inflammation or metaplasia were included in the corresponding squamous or columnar normal category.

TABLE 3
Reflectance Data Set by Histopathologic Category

| | Diagnostic Class | | | | | |
|---|---|---|---|---|---|---|
| | SN | CN | HPV | CIN 1 | CIN 2 | CIN 3/CIS |
| Number of sites | 227 | 18 | 52 | 9 | 3 | 15 |

Reflectance Spectra

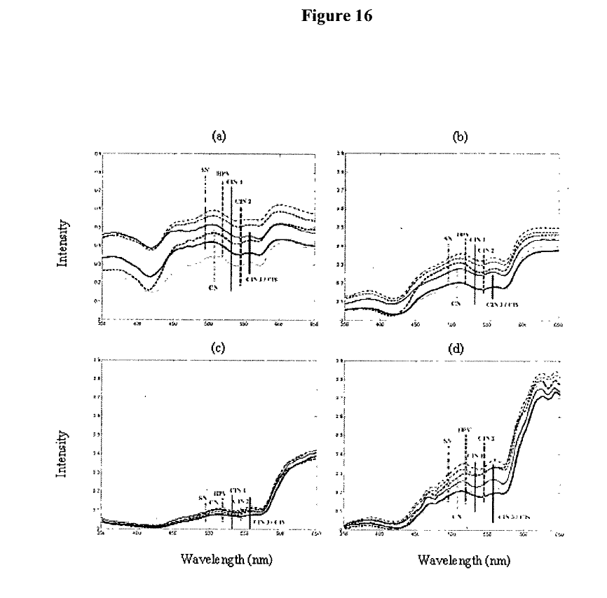

Average reflectance spectra for each diagnostic category for the four different source-detector separation positions are shown in FIG. 16. Each position (0, 1, 2, and 3) corresponds to an increasingly greater source-detection separation, which probes increasingly greater tissue depth. All spectra show valleys due to hemoglobin absorption at 420 nm, 542 nm, and 577 nm. As source-detector separation increases, the relative level of elastic scattering in the longer wavelengths increases due to increased penetration depth of light. Average spectra of each diagnostic class differ for all source-detector separations. At all separations, the mean reflectance of squamous normal tissues is most intense; there is gradual decrease in mean reflectance intensity from HPV to CIN 1 to CIN 2 to CIN 3, and the mean reflectance of columnar normal tissues is least intense. The greatest separation between average spectra of different diagnostic classes occurs at position 0.

Statistical Analysis

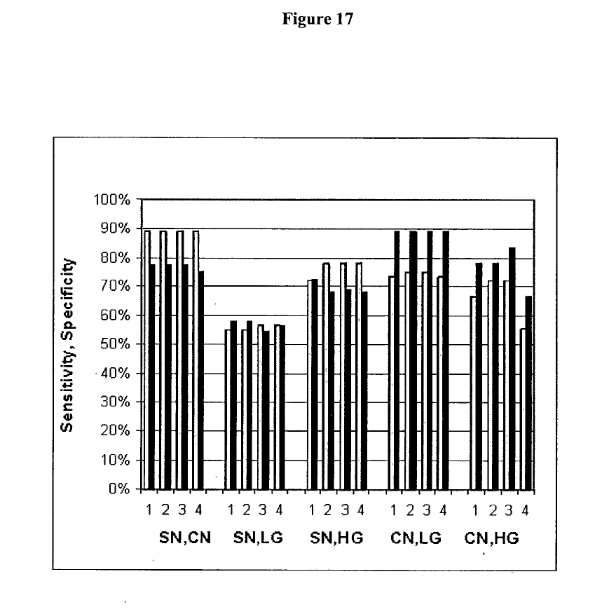

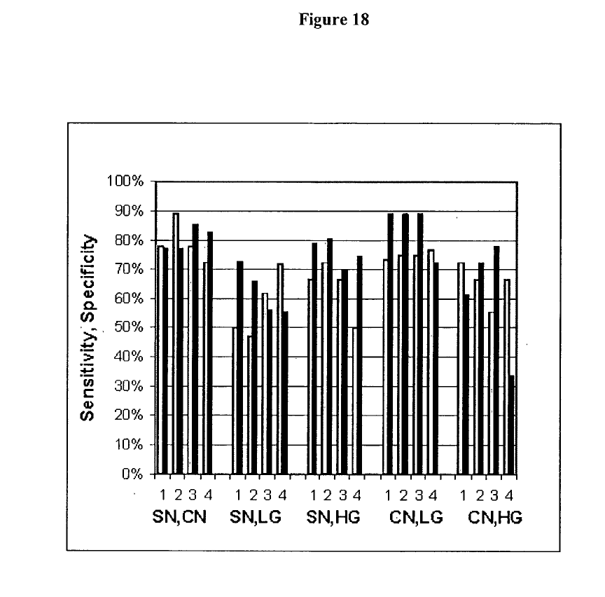

FIG. 17 shows the sensitivity and specificity for the best performing combination of one, two, three, and four source-detector separations at an ESL of 65%. Results are shown separately for each pairwise combination of diagnostic categories. The diagnostic performance is high when using only a single separation, and does not noticeably increase when data from additional separations is included. Best performance is obtained when discriminating between SN vs. CN, with sensitivity of 89% and specificity of 77%, and CN vs. LGSIL, with sensitivity of 75% and specificity of 89%. It is most difficult to discriminate between SN vs. LGSIL. Increasing the ESL from 65% to 95% does not result in an increase in performance (FIG. 18).

Table 4 gives the sensitivities and specificities for the combinations of source-detector separations that yielded the best performance. Many combinations of positions gave equally good results for discrimination of SN vs. CN and CN vs. LGSIL. Positions 0 and 1 appear in all of the optimal combinations for discriminating between tissue types, except SN vs. HGSIL.

TABLE 4

| | | | | # of | |
|---|---|---|---|---|---|
| | | | | Source- | Corresponding |
| Diagnostic | | | | Detector | Source- |
| Pairwise | Sensi- | Speci- | | Positions in | Detector |
| Combination | tivity | ficity | ESL | Combination | Positions |
| SN vs CN | 89% | 77% | 0.65 | 1 | 0 |
| | 89% | 77% | 0.65 | 2 | 0, 1 |
| | 89% | 77% | 0.65 | 3 | 0, 1, 2 |
| | 89% | 77% | 0.75 | 1 | 0 |
| | 89% | 77% | 0.75 | 2 | 0, 1 |
| | 89% | 77% | 0.75 | 3 | 0, 1, 2 |
| | 89% | 77% | 0.85 | 1 | 0 |
| | 89% | 77% | 0.85 | 2 | 0, 1 |
| | 89% | 77% | 0.85 | 3 | 0, 1, 2 |
| | 89% | 77% | 0.95 | 3 | 0, 1 |
| SN vs LG | 72% | 56% | 0.95 | 4 | 0, 1, 2, 3 |
| SN vs HG | 72% | 81% | 0.95 | 2 | 2, 3 |
| CN vs LG | 75% | 89% | 0.65 | 2 | 0, 1 |
| | 75% | 89% | 0.65 | 3 | 0, 1, 2 & 0, 1, 3 |
| | 75% | 89% | 0.75 | 2 | 0, 1 |
| | 75% | 89% | 0.75 | 3 | 0, 1, 2 & 0, 1, 3 |
| | 75% | 89% | 0.85 | 2 | 0, 1 |
| | 75% | 89% | 0.85 | 3 | 0, 1, 2 & 0, 1, 3 |
| | 75% | 89% | 0.95 | 2 | 0, 1 |
| | 75% | 89% | 0.95 | 3 | 0, 1, 3 |
| CN vs HG | 72% | 83% | 0.65 | 3 | 0, 1, 3 |
| | 72% | 83% | 0.75 | 3 | 1, 2, 3 |

Discussion

In the study of the diagnostic potential of reflectance spectroscopy, cervical in vivo measurements were obtained at four distinct source-detector separation positions. Mahalanobis distance classification was used to determine which source-detector separations contained the most diagnostically useful information. Results showed the sensitivity and specificity to be high when using a single source-detector separation at the lowest level of eigenvector significance considered, and do not noticeably increase when data from additional separations or ESLs are included. Furthermore, the smaller source-detector separations of positions 0 and 1 appear more frequently than the greater separations of positions 2 and 3 in the positional combinations that performed best. Positions 0 and 1 seem effective for all pairwise discrimination except SN vs. HGSIL.

The results show that HGSIL can be discriminated from squamous and columnar normal tissue with high sensitivity (72%, 72%) and high specificity (81%, 83%). These results indicate slightly lower sensitivity and improved specificity for discrimination of SN from HGSIL than determined in a previous study, where sensitivity of 82% and specificity of 67% were obtained using analysis of a single reflectance spectrum. Furthermore, it was found that LGSIL could be separated from columnar normal tissue with similar high sensitivity (75%) and specificity (89%); however, discrimination of LGSIL and squamous normal tissue was more difficult using reflectance spectroscopy in this study. In all cases, the specificity associated with reflectance spectroscopy was significantly higher than that previously reported for colposcopy (48%), while sensitivity was somewhat lower than that reported for colposcopy (96%). The sensitivities and specificities reported for reflectance spectroscopy here are based on leave-one-out cross-validation. Ideally, separate training and validation sets should be used to provide unbiased estimates of algorithm performance; however, previous work using fluorescence spectroscopy has shown that cross validation provides a good estimate of the performance with a separate validation set.

Given the single-cell epithelial thickness of columnar tissue, it is not surprising that its mean reflectance intensity is lower than the multi-layered squamous tissues at the various normal and dysplastic states. The single-layer epithelium allows for more light to reach and be absorbed by hemoglobin in the stromal tissue. All other diagnostic classes are multi-layered squamous epithelium and, as such, show a stronger mean reflectance intensity, which decreases with progression of disease. This is consistent with the results of Dellas, who found increased angiogenesis with increased severity of precancer. Using a Monte Carlo model of light transport in normal cervical epithelium, it was estimated that at 420 nm fluorescence excitation, the average penetration depth of photons detected at position 0 is 450 μm and at position 3 is 550 μm. Similarly, at 500 nm, the average penetration depth of photons detected at position 0 is 680 μm and at position 3 is 1180 μm. These simulations assumed an epithelial thickness of 350 μm and values of μ_{s,eptithelium}=105 cm⁻¹, μ_{a,eptithelium}=3 cm⁻¹, μ_{s,stroma}=280 cm⁻¹, μ_{a,stroma}=40 cm⁻¹ at 420 nm and μ_{s,eptithelium}=82 cm⁻¹, μ_{a,eptithelium}=2 cm⁻¹, μ_{s,stroma}=230 cm⁻¹, μ_{a,stroma}=4 cm⁻¹ at 500 nm. In these simulations, the anisotropy factor (g) of the epithelium and stroma were 0.95 and 0.88, respectively. The numerical aperture (NA) of the optical fiber was 0.22. The refractive index of the epithelium and the stroma were assumed to be 1.4. The code was verified with the results from Welch et al. for the two-layer model and with Mourant et al. for detector modeling. By keeping track of the photon position in tissue after each scattering event, the maximum depth the photon reaches can be calculated.

### EXAMPLE 3

### Materials and Methods: Fluorescence Only

Materials

During colposcopy, two colposcopically normal sites and one colposcopically abnormal site were chosen by the physician or nurse colposcopist, and fluorescence EEMs were measured from these three sites. It was noted whether these sites corresponded to squamous or columnar epithelium or the transformation zone.

Following fluorescence measurement, each site was biopsied and submitted for histopathologic diagnosis. Each Papanicolaou smear was read by the cyto-pathologist assigned to the case that day, and was subsequently reviewed by the study cyto-pathologist. Discrepant cases were reviewed a third time for consensus diagnosis by the study cytologist. Each biopsy was read by the pathologist assigned to the case that day, and was subsequently reviewed by the study histopathologist. Again, discrepant cases were reviewed a third time for consensus diagnosis by the study histopathologist. Standard diagnostic criteria were used and consensus diagnostic categories included: normal squamous epithelium (SN), normal columnar epithelium (CN), HPV infection (HPV), grade 1 cervical intraepithelial neoplasia (CIN 1), CIN 2, and grade 3 cervical intraepithelial neoplasia (CIN 3). For initial analysis, HPV infection and CIN 1 were grouped together as LGSIL, and CIN 2 and CIN 3 were grouped together as HGSIL.

Instrumentation

The spectroscopic system used to measure fluorescence EEMs has been described in detail previously. Briefly, the system measures fluorescence emission spectra at 16 excitation wavelengths, ranging from 330 nm to 480 nm in 10 nm increments with a spectral resolution of 5 nm. The system incorporates a fiber optic probe, a Xenon arc lamp coupled to a monochromator to provide excitation light and a polychromator and thermo-electrically cooled CCD camera to record fluorescence intensity as a function of emission wavelength. The fiber optic probe consists of 25 excitation fibers and 12 collection fibers, arranged randomly on a 2-mm diameter quartz fiber at the tip.

Measurements

As a negative control, a background EEM was obtained with the probe immersed in a non-fluorescent bottle filled with distilled water at the beginning of each day. Then a fluorescence EEM was measured with the probe placed on the surface of a quartz cuvette containing a solution of Rhodamine 610 (Exciton, Dayton, Ohio) dissolved in ethylene glycol (2 mg/mL) at the beginning of each patient measurement.

To correct for the non-uniform spectral response of the detection system, the spectra of two calibrated sources were measured at the beginning of the study; in the visible an NIST traceable calibrated tungsten ribbon filament lamp was used and in the UV a deuterium lamp was used (550C and 45D, Optronic Laboratories Inc, Orlando, Fla.). Correction factors were derived from these spectra. Dark current subtracted EEMs from patients were then corrected for the non-uniform spectral response of the detection system. Variations in the intensity of the fluorescence excitation light source at different excitation wavelengths were corrected using measurements of the intensity at each excitation wavelength at the probe tip made using a calibrated photodiode (818-UV, Newport Research Corp.).

Before the probe was used it was disinfected with Metricide (Metrex Research Corp.) for 20 minutes. The probe was then rinsed with water and dried with sterile gauze. The disinfected probe was guided into the vagina and its tip positioned flush with the cervical epithelium. Then fluorescence EEMs were measured from the three cervical sites. Acetic acid, which enhances the optical differences between normal and dysplastic tissue, was applied to the cervical epithelium prior to the placement of the probe. Measurement of each EEM required approximately two minutes.

Data Analysis

All spectra were reviewed by three investigators blinded to the pathologic results (SKC, UU and RRK) prior to analysis. Spectra which indicated evidence of instrument error or probe slippage were discarded from further analysis.

Fluorescence data were analyzed to determine which excitation wavelengths contained the most diagnostically useful information and to estimate the performance of diagnostic algorithms based on this information. Initially, algorithms that discriminated between all pair-wise combinations of diagnostic categories were explored (Table 5).

TABLE 5

| Class 1 | Class 2 |
|---|---|
| Squamous normal | Columnar normal |
| Squamous normal | LG-SIL |
| Columnar normal | LG-SIL |
| Squamous normal | HG-SIL |
| Columnar normal | HG-SIL |

In comparing all pairs of diagnostic classes, it can be determined which categories differ spectroscopically and assess where these differences are greatest. This information can then be used to develop multi-step algorithms to determine the tissue type of an unknown sample based on its fluorescence spectrum. For this purpose, an algorithm based on multivariate discriminant techniques which selects a subset of spectra at excitation wavelengths that perform best from all possible combinations of emission spectra at all excitation wavelengths was developed. The algorithm, described in detail below, consists of the following major steps: (1) data pre-processing to reduce inter-patient variations, (2) data reduction to reduce the dimensionality of the data set, (3) classification to classify the two given classes with maximum diagnostic performance and minimal likelihood of over-training in a training set, (4) evaluation of these algorithms using the technique of cross-validation.

Fluorescence data from a single measurement site is represented as a matrix containing calibrated fluorescence intensity as a function of excitation and emission wavelength. Columns of this matrix correspond to emission spectra at a particular excitation wavelength; rows of this matrix correspond to excitation spectra at a particular emission wavelength. Each excitation spectrum contains 16 intensity measurements ranging from 330 nm to 480 nm in 10 nm increments; each emission spectrum contains between 50 and 130 intensity measurements ranging between 380 nm and 910 nm in 5 nm increments, depending on excitation wavelength. Finally, emission spectra were truncated at emission wavelength of 700 nm to eliminate the highly variable background due to room light present above 700 nm. Most multivariate data analysis techniques require vector input, so prior to analysis the column vectors containing the emission spectra at excitation wavelengths selected for evaluation were cropped to discard the noisy tails and then were concatenated into a single vector.

Therefore, a pre-processing method was utilized to reduce large patient-to-patient variations in intensity that can be greater than the differences between histopathologic categories, the inter-patient variations, while preserving inter-category differences: each emission spectrum in the concatenated vector was normalized to its respective maximum intensity.

PCA (principal component analysis) was performed on the entire dataset for dimensionality reduction. First, an input matrix was created for each excitation wavelength combination by placing the concatenated spectrum vector from each sample in rows. The eigenvectors of the corresponding co-variance matrix were then calculated, yielding the principal components. Eigenvectors accounting was used for 65, 75, 85, and 95% of the total variance to investigate the effect of ESL (eigenvector significance level) on the algorithm performance. The ESL represents the fraction of the total variance of the dataset accounted for by the linear combination of the first n eigenvectors. Principal component scores associated with these eigenvectors were calculated for each sample.

Classification functions were then formed to assign a sample to one of the two given classes. The classification was based on the Mahalanobis distance, which is a multivariate measure of the separation of a data point from the mean of a dataset in n-dimensional space. The multivariate distance between the sample to be classified and the means of the two possible classification groups was calculated; the sample was then assigned to the group that it was closest to in this multivariate space.

The performance of classification depends on the principal component scores included for analysis. From the available pool of eigenvectors at ESLs of 65, 75, 85, and 95%, the single principal component score yielding the best initial performance was identified, and then the principal component score that improved this performance most was selected. This process was repeated until performance was no longer improved by the addition of principal component scores, or all the available scores were selected.

The sensitivity and specificity of the algorithm at each excitation wavelength combination were then evaluated relative to diagnosis based on histopathology. Overall diagnostic performance was evaluated as the sum of the sensitivity and the specificity, thus minimizing the number of misclassifications. The risk of overtraining was assessed for each of the excitation wavelength combinations by comparing the training set performance to the performance of an algorithm developed from the same dataset after the diagnosis corresponding to each sample had been randomized. The number of samples in each class was preserved during diagnosis randomization. This provides a dataset with the same variance structure as the original dataset but where the diagnostic performance is not expected to exceed that of chance. Diagnostic algorithms were then developed based on the randomized diagnoses. Random diagnoses were assigned 50 times for each wavelength combination and the average of the sensitivities and the specificities of the 50 cases were calculated. Ideally for completely normally distributed data, the sum of the sensitivity and specificity should be 1 for randomized diagnosis at all levels of training significance. However, if overtraining occurs, this sum will be greater than one. Combinations of emission spectra from one up to four excitation wavelengths were considered. Limiting the device to four wavelengths allows for construction of a reasonably cost-effective clinical spectroscopy system. To identify the optimal combination of excitation wavelengths, all possible combinations of up to four wavelengths chosen from the 16 possible excitation wavelengths were evaluated. This equated to 16 combinations of one, 120 combinations of two, 560 combinations of three, and 1,820 combinations of four excitation wavelengths, for a total of 2,516 combinations. The top-25 wavelength combinations were then ranked based in order of the increase in performance between the training set performance with the correct histopathologic diagnoses and the training set performance with random assignment of diagnosis. This method allows the top wavelength combinations to be ranked in order of their robustness, or lack of propensity to overtrain.

These estimates of algorithm performance are biased since they are based on the training set used to develop the algorithm. An unbiased performance estimate must be made to assess the true potential of each of the top-25 wavelength combinations. The effects of overtraining in performance estimation can be minimized by using separate training and validation sets, or by using the method of cross-validation. In the cross-validation method, a single data from the whole dataset is temporarily removed from the training dataset and the classification algorithm is developed using the remaining dataset as training set. The new classification algorithm is applied to the held out data. Each sample in the dataset was used as the test data in turn, and the sensitivity and specificity were calculated by comparing the classification result of each sample to histopathologic diagnosis. Among the top-25 wavelength combinations, results from top 10 combinations based on the sum of the cross-validated sensitivity and cross-validated specificity are presented in this paper.

### EXAMPLE 4

### Results: Flourescence Only

A total of 373 EEMs from 147 patients were analyzed in this study. Of the 373 EEMs reviewed, 22 were identified as defective for analysis due to instrument error (10 sites) and probe movement (12 sites), and were discarded. Of the 351 remaining EEMs, 233 sites were normal squamous sites, 23 were columnar sites, 64 were LGSILs and 31 were HGSILs. Of the 64 LGSIL sites, 46 were HPV and 18 were CIN 1. Of the 31 HGSIL sites, 12 were CIN 2 sites, 11 were CIN 3, 8 were carcinoma in situ (CIS). Of the 233 squamous normal sites 107 sites had inflammation and/or metaplasia. 40 showed inflammation only and 14 showed metaplasia only. The rest 53 sites had both inflammation and metaplasia. Of the 23 columnar normal sites, 12 showed inflammation only and 11 showed both inflammation and metaplasia. None of the columnar sites had just metaplasia. The composition of data used for analysis is summarized in Table 6.

TABLE 6

| Histopathologic category | Number of samples | Composition | |
|---|---|---|---|
| Squamous normal | 233 | | |
| Columnar normal | 23 | | |
| LG-SIL | 64 | HPV: 46 | CIN 1: 18 |
| HG-SIL | 31 | CIN 2: 12 | CIN 3+: 19 |

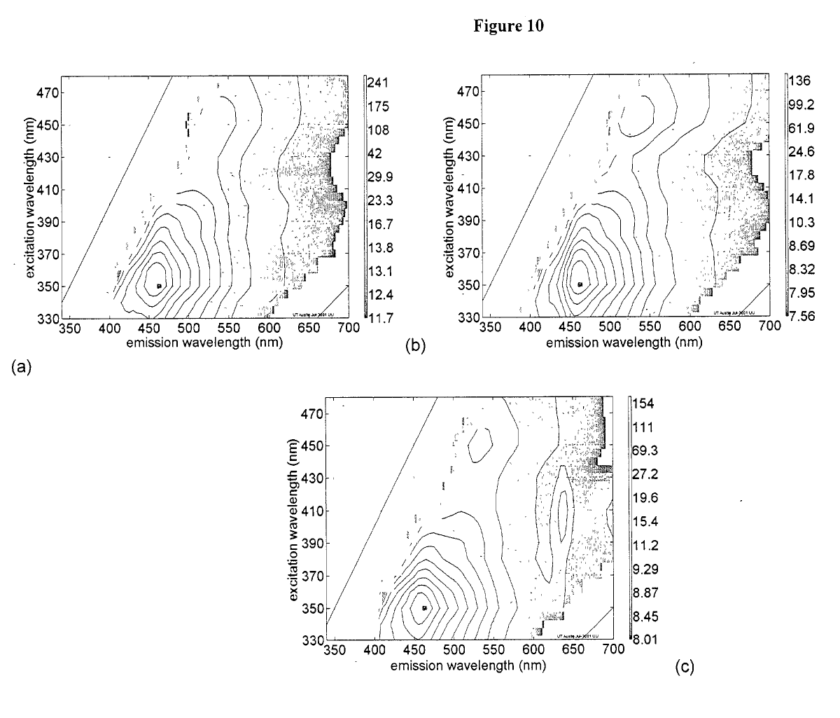

FIG. 10 shows typical fluorescence EEMs from different sites in the same patient, including a normal squamous site, a normal columnar site and a site with HGSIL. The fluorescence EEMs are plotted as topographical maps, with excitation wavelength on the ordinate and emission wavelength on the abscissa. Contour lines connect points of equal fluorescence intensity. Several excitation-emission maxima are present; the peak at 350 nm excitation, 450 nm emission is consistent with emission of the co-factor NADH as well as collagen crosslinks. A less apparent shoulder at 370 nm excitation, 525 nm emission is consistent with emission of the co-factor FAD. The peak at 450 nm excitation, 525 nm emission is consistent with the co-factor FAD as well as structural protein fluorescence. In addition, fluorescence of endogenous porphyrins is present in the EEM of the HGSIL, with excitation maxima at 410 nm and emission maxima at 630 and 690 nm. Tissue vascularity can influence fluorescence spectra, when hemoglobin absorbs fluorescent light at 420, 540 and 580 nm, producing valleys in the EEMs parallel to the excitation and emission wavelength axes as seen in all three EEMs.

TABLE 7

| Excitation | Cross-validated | Cross-validated |
|---|---|---|
| Wavelength (nm) | Sensitivity | Specificity |
| 390 | 0.74 | 0.74 |
| 350 | 0.71 | 0.73 |
| 400 | 0.68 | 0.76 |
| 440 | 0.71 | 0.72 |
| 340 | 0.71 | 0.71 |
| 380 | 0.68 | 0.68 |
| 330 | 0.65 | 0.71 |
| 430 | 0.65 | 0.70 |
| 370 | 0.65 | 0.70 |
| 420 | 0.55 | 0.78 |
| MEAN | 0.67 | 0.72 |

Table 7 shows the cross-validated sensitivity and specificity for algorithms based on top 10 performing single excitation wavelengths. An ESL of 75% was used; however, similar results were obtained at all ESLs. The excitation wavelengths are listed in descending order of the sum of the cross-validated sensitivity and specificity. Tables 7 and 8 show the cross-validated sensitivity and specificity for algorithms based on the top 10 performing combinations of 2 and 3 wavelength combinations, respectively. Again, an ESL of 75% was used, but similar results were obtained at all ESLs. The excitation wavelength combinations in each table are listed in descending order of the sum of the cross-validated sensitivity and specificity. Table 7 shows that, while the four top excitation wavelengths have similar performance, sensitivity and specificity drop for the remaining single excitation wavelengths. However, most of the top 10 combinations of two excitation wavelengths (Table 8) and all of the top 10 combinations of three excitation wavelengths (Table 9) have similar performance. Interestingly, the four excitation wavelengths which give the best performance in Table 7 appear in many of the top 10 combinations identified in Table 8 and Table 9.

TABLE 8

| Excitation | Excitation | | |
|---|---|---|---|
| Wavelength 1 | Wavelength 2 | Cross-validated | Cross-validated |
| (nm) | (nm) | Sensitivity | Specificity |
| 350 | 430 | 0.68 | 0.86 |
| 430 | 460 | 0.68 | 0.82 |
| 330 | 410 | 0.68 | 0.79 |
| 330 | 400 | 0.74 | 0.72 |
| 330 | 420 | 0.74 | 0.72 |
| 330 | 450 | 0.65 | 0.82 |
| 330 | 430 | 0.74 | 0.72 |
| 360 | 410 | 0.65 | 0.81 |
| 420 | 460 | 0.74 | 0.71 |
| 430 | 450 | 0.65 | 0.80 |
| MEAN | | 0.70 | 0.78 |

TABLE 9

| Excitation | Excitation | Excitation | Cross-validated | Cross-validated |
|---|---|---|---|---|
| Wavelength 1 (nm) | Wavelength 2 (nm) | Wavelength 3 (nm) | Sensitivity | Specificity |
| 420 | 430 | 460 | 0.71 | 0.79 |
| 330 | 460 | 470 | 0.65 | 0.85 |
| 330 | 420 | 470 | 0.65 | 0.85 |
| 340 | 380 | 420 | 0.68 | 0.79 |
| 330 | 340 | 420 | 0.68 | 0.79 |
| 340 | 420 | 470 | 0.74 | 0.72 |
| 350 | 430 | 440 | 0.74 | 0.72 |
| 350 | 440 | 470 | 0.74 | 0.72 |
| 340 | 420 | 460 | 0.74 | 0.72 |
| 330 | 400 | 470 | 0.74 | 0.71 |
| MEAN | | | 0.71 | 0.77 |

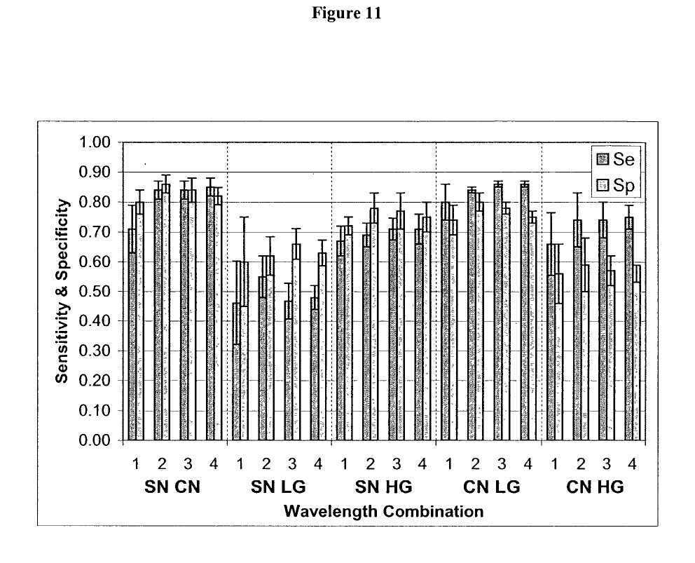

FIG. 11 illustrates the effect of increasing the number of excitation wavelengths for algorithms that classify the important histopathological categories in the cervix. FIG. 11 shows the mean and the standard deviation of the cross-validated sensitivities and specificities from the top-10 excitation wavelength combinations for each pair of classes tested at an ESL of 75%. In general, the performance of the algorithms developed for a pair of classes improves when the number of excitation wavelengths used in the combination is increased from one to two; however, adding a third or a fourth excitation wavelength does not generally improve diagnostic performance. The algorithm performs well for separating SN vs. CN, SN vs. HGSIL, and CN vs. LGSIL. In contrast, lower performance is observed for separating SN vs. LGSIL and CN vs. HGSIL.

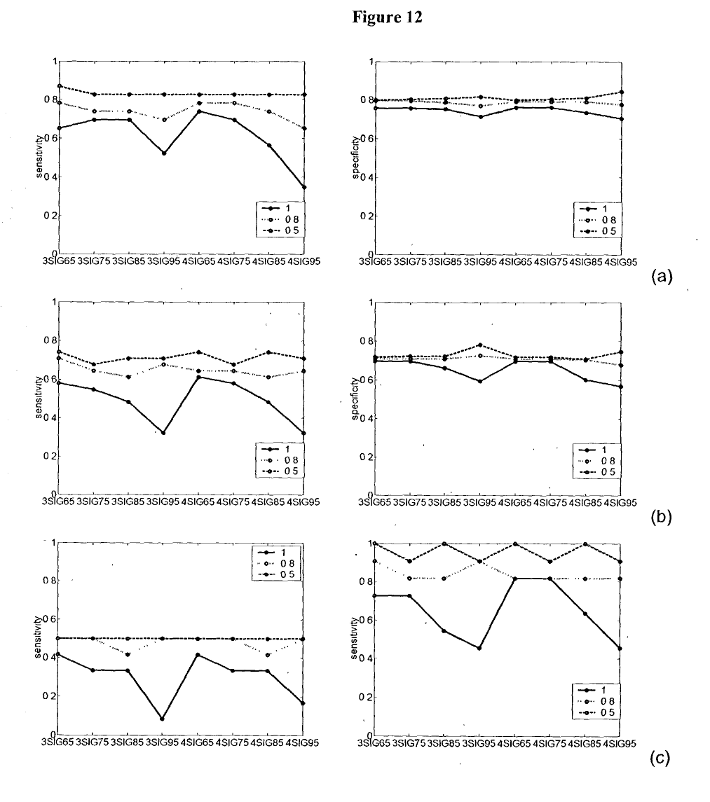

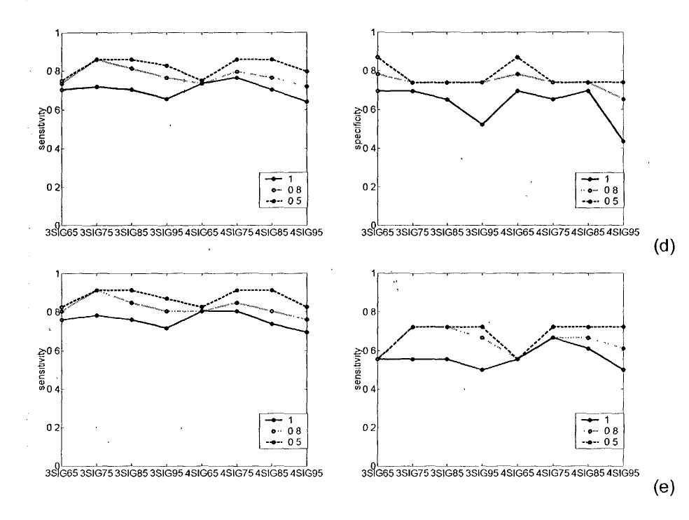

FIG. 12 illustrates the performance of ensemble classifiers based on the top-25 performing combinations of three and four excitation wavelengths at ESLs of 65%, 75%, 85% and 95%. FIG. 12A shows the sensitivity (left) and specificity (right) for ensemble classifiers to separate SN vs. CN. Performance of three types of ensembles are shown: in the first the sample was classified as CN if a majority of the 25 individual top performing excitation wavelength combinations indicated the sample was CN. Similarly, results from the classifiers which required 80% or 100% of the top performing 25 excitation wavelength combinations to classify the sample as CN are also shown. Again, as the number of excitation wavelengths is increased, little change in performance is seen. As the percentage of individual classifiers required to identify a sample as CN is increased from a majority to unanimous, little decrease is seen in the specificity, indicating that most excitation wavelength combinations yield the same classification. Finally, it is interesting to note that the sensitivity drops slightly at an ESL of 95% when all 25 individual classifiers are required to agree. This likely reflects an overtraining bias at this ESL.

FIG. 12B shows the performance of ensemble classifiers based on the top 25 performing combinations of three and four excitation wavelengths to separate SN from HGSILs. As the number of individual classifiers required to identify a sample as HGSIL increased from a majority to unanimous, sensitivity dropped while specificity remained fairly constant. Again, as the number of excitation wavelengths was increased from three to four, no significant increase in performance were observed. FIG. 12C shows the sensitivity of ensemble classifiers for CIN 2 (left) and CIN 3 (right). Sensitivity is much higher for CIN 3 than for CIN 2 for the three ensemble classifiers. This is consistent with the ability of the pathologist to identity CIN 3; inter-observer agreement is much higher for diagnosis of CIN 3 than for CIN 2.

Similarly, FIG. 12D shows the performance of ensemble classifiers which separate CN from LGSIL. In this case, neither sensitivity nor specificity varied substantially as the fraction of individual classifiers required to classify a sample as LGSIL was increased from 50% to 100%. Again, the diagnostic performance did not increase as the number of excitation wavelengths was increased from three to four. To explore whether performance varied for the two sub-categories which make up LGSIL (HPV infection and CIN 1), sensitivity for these two sub-categories were separately examined. Results are shown in FIG. 12E. The sensitivity for discriminating CN and HPV (left) was higher than that for CIN 1 (right), indicating that CN spectra more closely resemble those of tissue with CIN 1 than with HPV infection. Although the plots are not shown, the ensemble classifiers for discriminating LGSIL from SN shows similar results where addition of an excitation wavelength to 4 wavelength combinations do not increase performance. Investigation of the subcategories for LGSIL shows that difficulty of separating HPV from SN limits the performance.

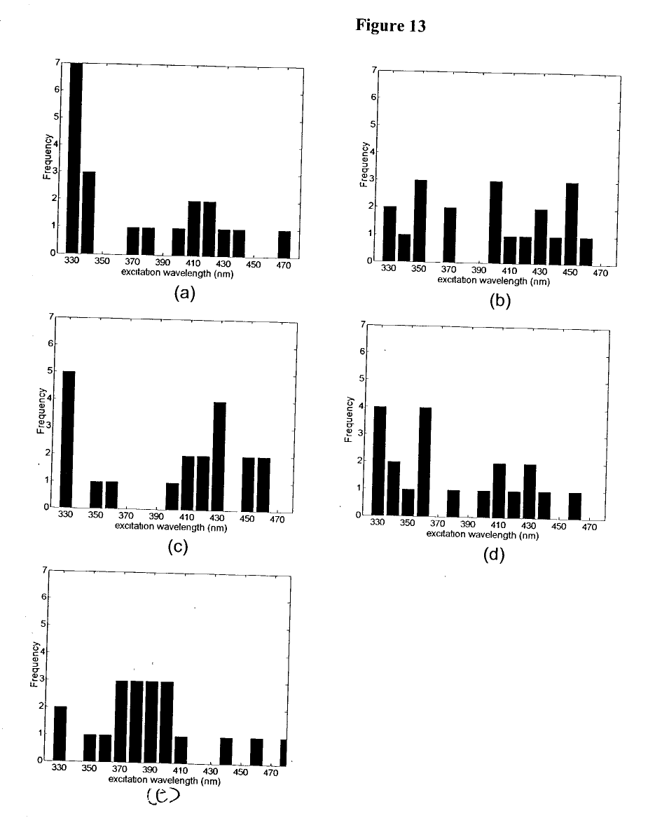

In order to select the wavelengths most promising for discrimination between histopathologic classes of cervical tissue, those excitation wavelengths which resulted in highest diagnostic performance were explored. Histograms were created indicating the frequency of occurrence of each excitation wavelength in the top 10 performing combinations of two excitation wavelengths at an ESL of 75% for all pairwise discriminations. Results are shown in FIG. 13. FIG. 13A shows the histogram for discrimination between SN and CN, and indicates that 330-340 nm occurs most frequently, and wavelengths of 410 and 420 nm also occur frequently. FIG. 13B shows the histogram for discrimination between SN and LGSIL. Again, 330-350 nm excitation occurs frequently, as well as 400-450 nm excitation. FIGS. 13C and 13D show similar wavelengths are useful for discriminating between SN and HGSIL as well as between CN and LGSIL. FIG. 13E shows that excitation wavelengths between 370-400 nm are most useful for discriminating CN and HGSIL.

### EXAMPLE 5

### Materials and Methods: Reflectance and Flourescence

Instrumentation

The spectroscopic system used to measure reflectance spectra has previously been described in detail. Briefly, the system incorporates three main components: (1) a Xenon arc lamp used to provide reflectance broadband illumination and coupled to a monochromator to provide fluorescence excitation light; (2) a fiber-optic probe that directs the light to tissue and collects diffusely reflected and fluorescent emission light; and (3) an optical assembly with a polychromator and thermo-electrically cooled CCD camera to record the spectral data. FIG. 14 illustrates the system.

The probe, illustrated in FIG. 15, utilizes a fiber optic bundle for fluorescence measurement in the core surrounded by nine spatially separated reflectance optical fibers, placed in direct contact with the tissue [200 μm diameter fibers, numerical aperture (NA)=0.2]. The fluorescence bundle consists of a random arrangement of 25 illumination and 12 collection fibers—a 15 mm long quartz mixing element [200 μm diameter, (NA)=0.2] at the distal end of the bundle separates the fibers from direct contact with the measurement tissue, ensuring all fibers in the bundle illuminate and collect fluorescence from the same area of tissue. Fluorescence excitation wavelengths range from 330 nm to 480 nm in 10 nm increments and each emission spectra has a spectral resolution of 5 nm. Of the nine reflectance fibers, one excitation fiber provides broadband illumination and eight reflectance collection fibers collect diffusely reflected light, at four different source-detector separations (position 0: 250 μm separation, position 1: 1.1 mm separation, position 2: 2.1 mm separation, position 3: 3.0 mm separation) for probing increasingly greater tissue depth. A single spectroscopic measurement consists of fluorescence and reflectance spectra measured in sequence in a two-minute interval.

Clinical Measurements

Following colposcopic examination, but prior to biopsy, a fiber optic probe was advanced through the speculum and placed in gentle contact with the cervix. Both fluorescence and reflectance measurements were obtained from up to two colposcopically abnormal cervical sites, one colposcopically normal cervical site covered with squamous epithelium, and if visible, one colposcopically normal cervical site covered with columnar epithelium. Following spectroscopic measurements, all sites interrogated with the fiber-optic probe were biopsied.

Within two hours of each patient measurement, spectra from reflectance and fluorescence standards were measured. As a positive control for reflectance measurements, reflectance spectra were measured from a one cm pathlength cuvette containing a suspension of 1.02 μm diameter polystyrene microspheres (6.25% by vol.), to mimic the optical properties of tissue. Fluorescence spectra were measured from a solution of Rhodamine 610 (Exciton, Dayton, Ohio) dissolved in ethylene glycol (2 mg/ml) in a one cm pathlength cuvette, for positive control of fluorescence measurements. As a negative control, reflectance & fluorescence spectra were measured with the probe tip immersed in a large container of distilled water.

Biopsies were fixed and submitted for permanent section. Four-micron thick sections were stained with both hematoxylin and eosin (H&E) and Feulgen stained. Histologic diagnostic classification categories included normal tissues, HPV infection (HPV), grade 1 cervical intra-epithelial neoplasia (CIN 1), grade 2 cervical intra-epithelial neoplasia (CIN 2), grade 3 cervical intra-epithelial neoplasia (CIN 3), and carcinoma in situ (CIS). Normal tissues were divided into two categories based on colposcopic impression: normal squamous epithelium (SN) and normal columnar epithelium (CN). In accordance with the Bethesda system, HPV and CIN 1 were termed LGSILs and CIN 2, CIN 3, and CIS were termed HGSILs. The diagnostic categories SN, CN, LGSIL, and HGSIL were used in this analysis.

Data Processing & Statistical Analysis

Three investigators, blinded to the pathologic results, reviewed all spectra. Spectra indicating evidence of user or instrument error, such as probe slippage, were discarded from further analysis. Effects of the source spectrum, variations in the illumination intensity, and the wavelength-dependent response of the detection system, were corrected using the microsphere suspension reflectance standard and fluorescence correction factors. Reflectance spectra at each source-detector separation were normalized by the corresponding spectrum from the microsphere suspension. While this procedure results in spectra that describe the transport of light in tissue relative to that in microsphere suspension, the transport properties of microspheres are well known and can easily be described using Monte-Carlo based models. Variations in the illumination intensity of the light source at all excitation wavelengths were corrected with excitation illumination intensity measured at the probe tip using a calibrated photodiode (818-UV, Newport Research Corp.). To correct for the non-uniform spectral response of the detection system, the spectra of two calibrated sources were measured at the beginning of the study; in the visible a NIST traceable calibrated tungsten ribbon filament lamp was used and in the UV a deuterium lamp was used (550C and 45D, Optronic Laboratories Inc, Orlando, Fla.). Correction factors were derived from these spectra.

Reflectance data from a single measurement site are represented as a matrix containing calibrated reflectance intensity as a function of source-detector separation position and emission wavelength. Spectra from the four positions are column vectors containing 121 intensity measurements corresponding to emission wavelengths from 355 nm to 655 nm in 2.5 nm increments. Fluorescence data from a single measurement site is represented as an excitation-emission matrix (EEM), where calibrated fluorescence intensity is expressed as a function of excitation and emission wavelength. Columns of this matrix correspond to emission spectra at each excitation wavelength, containing between 50 to 130 intensity measurements ranging from 380 nm to 910 nm emission in 5 nm increments. The excitation wavelengths range from 330 nm to 480 nm in 10 nm increments. Fluorescence emission spectra at wavelengths greater than 700 nm were truncated to eliminate the highly variable background present above 700 nm.

The reflectance and fluorescence spectra were then analyzed to determine which source-detector separations and excitation wavelengths, to be termed classification features, contained the most diagnostically useful information to separate each diagnostic category of tissue found in the cervix. An algorithm to separate each diagnostic category pairing was developed and evaluated relative to the gold standard of colposcopically directed biopsy. In comparing all pairs of diagnostic categories, it is possible to determine which categories differ spectroscopically and assess which classification features show the greatest differences. Algorithm development consisted of the following steps: (1) generation of data matrices corresponding to classification feature combinations to analyze; (2) data reduction using principal component analysis (PCA); (3) data classification with cross-validation using Mahalanobis distance.

To identify the optimal classification combination among four source-detector separations and sixteen excitation wavelengths, all possible combinations were evaluated when the twenty features are taken one, two, or three at a time. There were a total of 1350 combinations considered—twenty of one, 190 of two, and 1140 of three.

Prior to PCA, components of the measurement spectra corresponding to the classification features of a given combination were concatenated into a single vector. Data matrices for each pair-wise analysis were assembled from these vectors, where a row corresponds to the concatenated vector from a measurement site. Only the measurements from the pair being analyzed were assembled into a matrix. Eigenvectors of the corresponding covariance matrix were then calculated; those accounting for up to 65, 75, 85, and 95% of the total variance were retained for algorithm development. The fraction of the total variance accounted for were denoted as the eigenvector significance level (ESL). For each eigenvector, a principal-component score was calculated for each sample in the data matrix.

Classification functions were then formed to assign a sample to one of the two given diagnostic categories. Classification was based on the Mahalanobis distance, the multivariate measure of the separation of a data point from the mean of a dataset in n-dimensional space. The multivariate distance between the measurement to be classified and the means of the two histopathologic categories were calculated, and the sample was assigned to the group it was closest to in multivariate space.

The performance of classification depends on the principal-component scores included for analysis. From the available pool of eigenvectors at each ESL, the single principal-component score yielding the best initial performance was identified, and then the score that improved this performance most was selected. This process was repeated until performance was no longer enhanced by the addition of principal components, or until all components were selected. The sensitivity and specificity of diagnostic algorithms for each classification feature combination were then evaluated relative to the histopathologic diagnosis—disease tissue was taken as the positive sample relative to either columnar or squamous normal tissue and columnar normal tissue was taken as the positive sample relative to squamous normal tissue.

In order to determine which combination of classification features were significant, the classification algorithm was trained using all the samples in the pair of diagnostic categories under consideration. The algorithm was then tested against its training sample set—each sample in the set was given a random diagnosis and the algorithm was run against the modified set. Randomized diagnosis is performed to reduce the risk of overtraining. All of the combinations were ranked with respect to the difference in performance between the true diagnosis and the mean of the 50 randomized diagnosis trials. The top 25 ranking combinations were then further evaluated. To further reduce the risk of overtraining, cross-validation was used to estimate algorithm performance of the top 25 combinations. In this process, a single sample from a whole data set is temporarily removed and a classification algorithm is developed using the remaining data. The algorithm is applied to the held-out data. Each sample in the dataset was held out in turn and the sensitivity and specificity were calculated by comparing the classification result of each sample to histopathologic diagnosis. Diagnostic performance of each classification feature combination therefore was evaluated as the sum of the cross-validated sensitivity and specificity.

### EXAMPLE 6

### Results: Reflectance and Flourescence

Data Set

The data consisted of a set of spectra from 324 sites from 161 patients that were deemed adequate for both reflectance and fluorescence analysis. Table 10 shows data sets by histopathologic category (colposcopically directed biopsy gold standard). Entries in Table 10 indicate the number of measurements within each diagnostic category for the data set. Tissues with acute or chronic inflammation or metaplasia were included in the corresponding squamous or columnar normal category.

TABLE 10

| | Diagnositic Class | | | | | | |
|---|---|---|---|---|---|---|---|
| | SN | CN | HPV | CIN 1 | CIN 2 | CIN 3/CIS | Total |
| Number of sites (161 patients) | 227 | 18 | 52 | 9 | 3 | 15 | 324 |

Reflectance Spectra

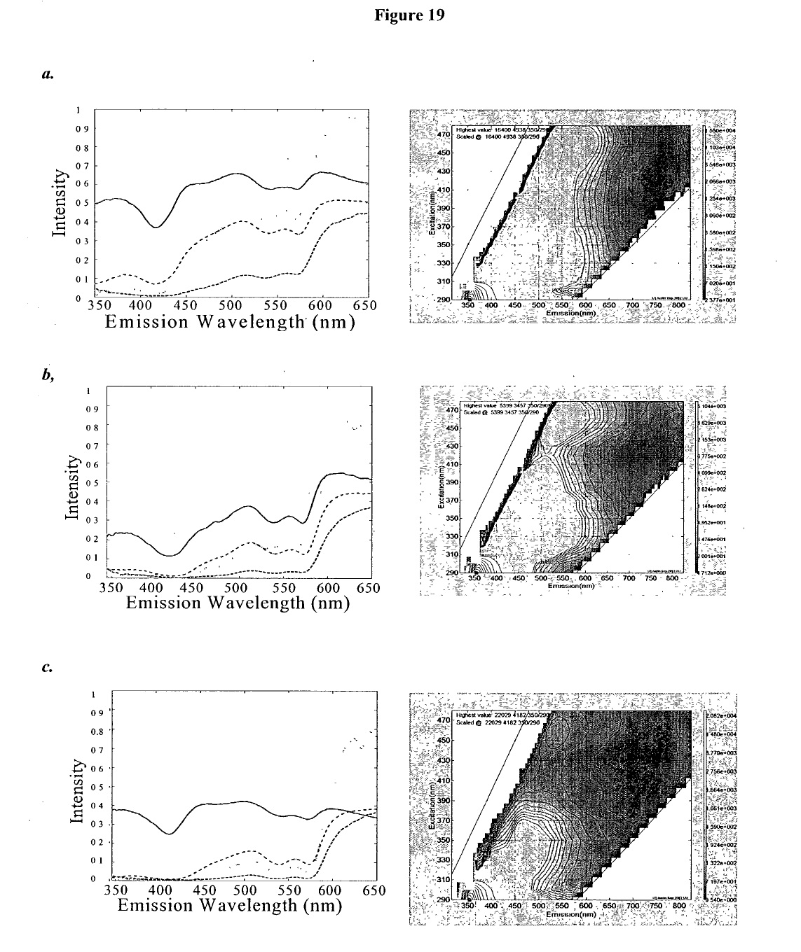

Typical reflectance and fluorescence spectra from three tissue measurement sites diagnosed as (a) normal squamous, (b) normal columnar, and (c) CIN 3/CIS are shown in FIG. 19. The reflectance spectra for each site, at the four different source-detector separation positions, are shown in the left column of FIG. 19. Positions 0, 1, 2, and 3 correspond to an increasingly greater source-detection separation, which probes increasingly greater tissue depth. All reflectance spectra show valleys due to hemoglobin absorption at 420 nm, 542 nm, and 577 nrm. As source-detector separation increases, the relative level of elastic scattering in the longer wavelengths increases due to increased penetration depth of light. In general, reflectance intensity decreases from SN tissue to abnormal tissue, with the most significant attenuation observed with HGSIL. Reflectance intensity from CN tissue is relatively low compared to that from SN tissue.

Fluorescence Spectra

The fluorescence EEM spectra are shown in the right column of FIG. 19. Fluorescence peaks from cofactors NADH, FAD, and structural proteins, as well as hemoglobin absorption valleys, are visible in the EEMs. Fluorescence from cofactor NADH induces a peak at 350 nm excitation/450 nm emission, while co-factor FAD induces a peak along 525 nm emission at both 350 nm and 450 nm excitation. Fluorescence from porphyrin, if present, appears as a peak at 410 nm excitation/630 nm emission. Absorption due to hemoglobin causes valleys parallel to the excitation and emission wavelength axes along 420 nm, 540 nm, and 580 nm.

Statistical Analysis

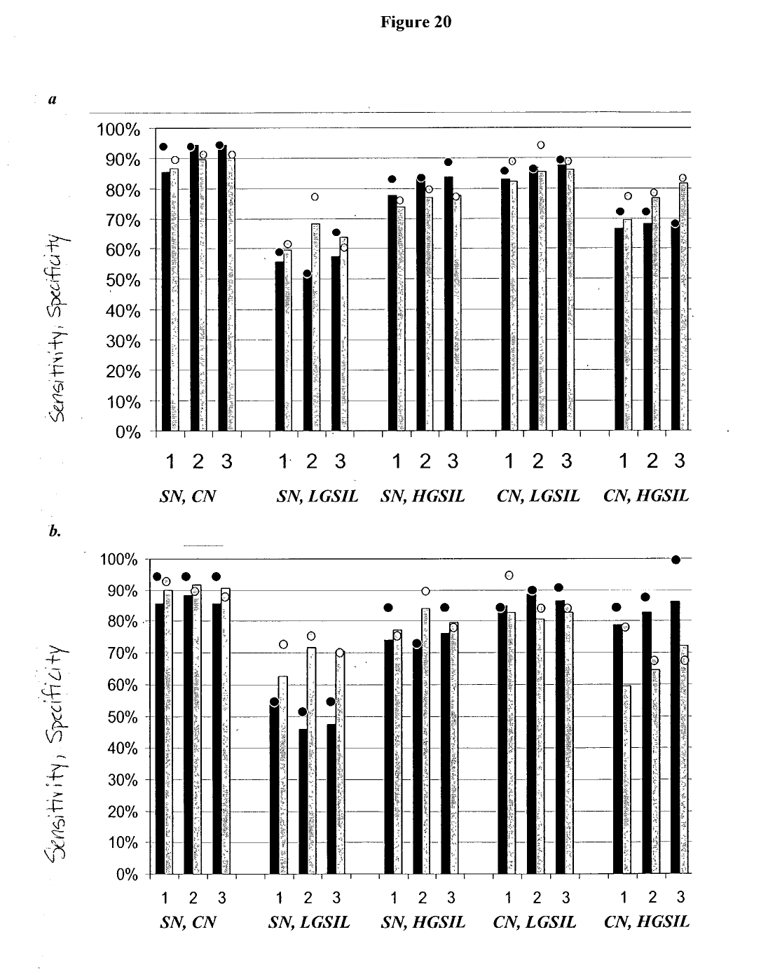

The average cross-validated sensitivity and specificity of the ten best-performing combinations of one, two, and three classification features, is indicated in FIG. 20. Results are shown separately for each diagnostic category pairing. FIG. 20A shows the performance at an ESL of 65%. The diagnostic performance is high when limited to the use of a single feature, and only small increases in performance are seen with each inclusion of an additional feature. Best performance is obtained when discriminating between SN and CN, reaching an average sensitivity of 94% and specificity of 90% with the use of two or three classification features. It is most difficult to discriminate between SN and LGSIL, where the best performance shows an average sensitivity of 58% and specificity of 64% with the use of three classification features. Dots indicate the sensitivity and specificity of the single best performing combination in the average for each analysis. Again, as seen with the average performance, increasing the number of features does not result in significant gains in diagnostic performance. Where the addition of features actually decreased performance, over-training of the data is the probable cause. Furthermore, increasing the ESL from 65% to 95% does not result in a noticeable increase in performance either (FIG. 20B).

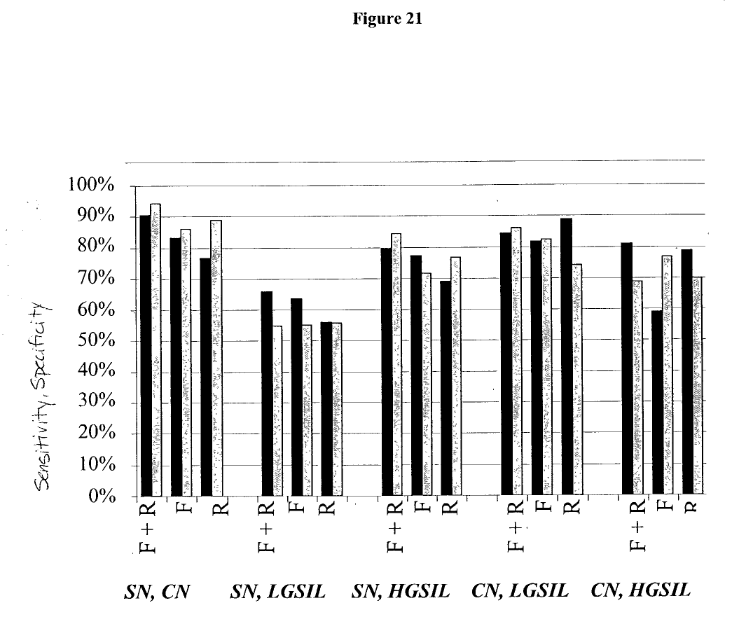

For each pair of diagnostic categories, FIG. 21 shows the average sensitivity and specificity of the five best performing combination of classification features, for analyses of reflectance spectra alone, fluorescence EEMs alone, and reflectance combined with fluorescence. In the reflectance alone analysis, up to four classification features were considered for each combination, while the fluorescence alone and the combined analyses were limited to up to three features in each combination. For all pairs of categories, the reflectance alone analysis indicated good diagnostic performance, with strong gains seen from the fluorescence alone analysis relative to the reflectance alone analysis. The addition of reflectance features to fluorescence features resulted in a modest improvement of discrimination performance, as indicated by the combined analysis.

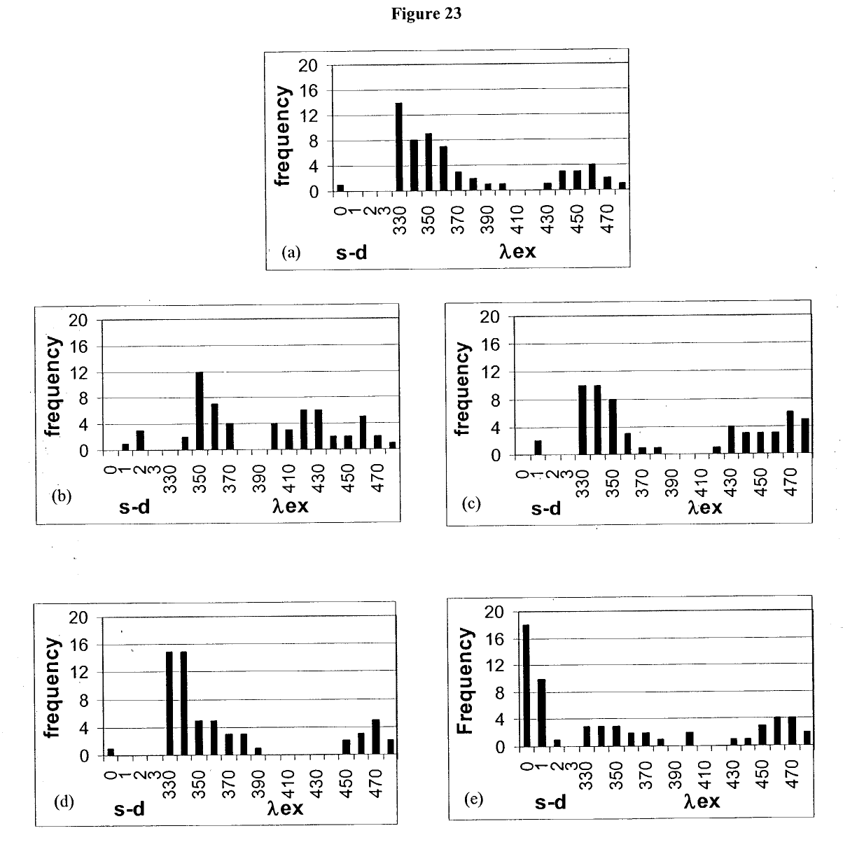

FIG. 23 shows the frequency with which each classification feature cumulatively appears within the ten best-performing feature combinations, for each one, two, and three combination of features when both fluorescence and reflectance are considered. Results are shown separately for each pair of diagnostic categories. The classification features most frequently included for discriminating between SN and CN are fluorescence excitation wavelengths 330-360 nm and 460 nm. Similarly, discrimination of SN from HGSILs and CN from LGSILs, fluorescence excitation wavelengths 330-350 nm with 470-480 nm and wavelengths 330-360 nm with 470 nm, respectively, predominate in occurrence. In the determination of SN tissue from LGSILs, fluorescence excitation wavelengths 350-360 nm and 460 nm, with the addition of wavelengths 420-430 nm, also frequently appear in the best performing feature combinations. Uniquely for discrimination of CN tissue from HGSILs. reflectance source-detector separations 0 and 1 occurred most frequently. In general, fluorescence excitation wavelengths 330-360 nm and 460-470 nm are significant in all pair-wise discrimination between diagnostic categories, though no classification feature appears to be singularly optimal for all discrimination. Reflectance features appear significant only in the discrimination of CN from HGSILs.

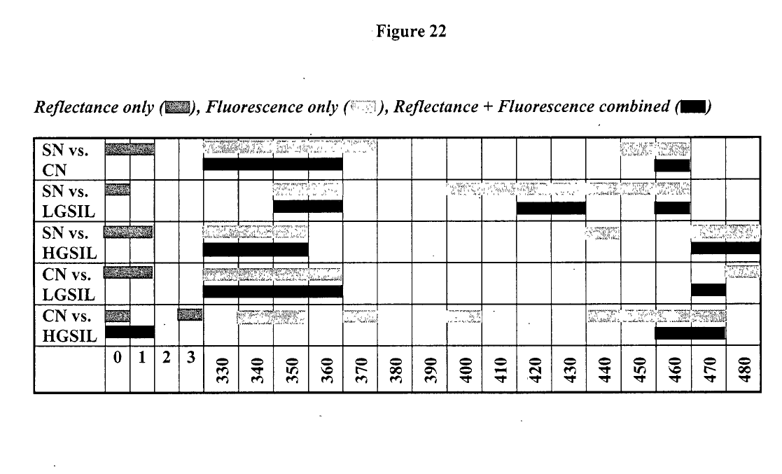

In FIG. 22, a comparison is made of the classification features that predominate, again in terms of relative maximum frequency of inclusion in the best performing feature combinations for pair-wise discrimination between diagnostic categories, for the three analyses considered—reflectance alone, fluorescence alone, and reflectance and fluorescence combined, all from identical patient-sites.

For all pair-wise fluorescence alone and combined analyses, only fluorescence excitation wavelengths in the regions of 330-360 nm and 460-470 nm appear significant—where significance is designated by a minimum of four occurrences and relative dominance amongst local features. The corresponding pair-wise reflectance alone analyses show source-detector separation positions 0 and 1 to be significant, but apparently lose importance when considered with the fluorescence features in the combination analysis, with the exception of CN vs. HGSIL. In the case of CN vs. HGSIL, reflectance source-detector separation positions 0 and 3 appear significant in the reflectance alone analysis and the fluorescence excitation wavelengths in the region of 360-400 nm appear significant in the fluorescence alone analysis. In contrast, the combined analysis identifies reflectance features 0 and 1 as significant and fluorescence features 460-470 nm as significant, both different from the features selected in the individual analyses. However, FIG. 23E shows that the difference in frequency between fluorescence excitation wavelengths in the 460-470 nm region and those in the 330-350 nm region to be very small. In fact, performance results show that combined analysis performs comparably when reflectance features 0 and 1 are combined with either the 330-350 nm or 460-470 nm region of fluorescence excitation wavelengths.

Furthermore to select the features that yield the best performance in differentiation between all diagnostic category pairings, the source-detector separations and excitation wavelengths were considered in terms of three and four feature sets (Table 11). For each three-feature set, the combination of features from the set that gave the best performance for each of the five diagnostic category pairings was determined. Overall best results from comparison of classification feature combinations, when taken in sets of three or of four features at a time, based on the sensitivity and specificity performance values obtained from pair-wise discrimination between diagnostic categories for an ESL=65% are shown.

TABLE 11
Typical ‘Four Feature Set’ Best Results
(330 nm, 360 nm, 430 nm, 470 nm shown)

| Diagnostic Pair | Sensitivity | Specificity | Combination |
|---|---|---|---|
| SN vs. CN | 94% | 91% | 330 nm, 470 nm |
| SN vs. LGSIL | 55% | 63% | 430 nm |
| SN vs. HGSIL | 83% | 80% | 330 nm, 430 nm |
| CN vs. LGSIL | 87% | 94% | 330 nm, 360 nm |
| CN vs. HGSIL | 72% | 78% | 470 nm |
| Averages | 78% | 81% | |

Typical ‘Three Feature Set’ Best Results
(330 nm, 430 nm, 470 nm shown)

| Diagnostic Pair | Sensitivity | Specificity | Combination |
|---|---|---|---|
| SN vs. CN | 94% | 91% | 330 nm, 470 nm |
| SN vs. LGSIL | 55% | 63% | 430 nm |
| SN vs. HGSIL | 83% | 80% | 330 nm, 430 nm |
| CN vs. LGSIL | 90% | 83% | 330 nm |
| CN vs. HGSIL | 72% | 78% | 470 nm |
| Averages | 79% | 79% | |

Overall performance of each three-feature set was calculated by the average of the sensitivities and specificities of the five determined combinations and the set of three features 5 that yielded the highest overall performance was selected. This analysis was done for each four-feature set as well. In the case of the four-feature set analysis, fluorescence excitation wavelengths 330 nm, 360 nm, 430 nm, and 470 nm were selected, giving a mean sensitivity of 78% and mean specificity of 81% for the five diagnostic category pairings. For the three-feature set analysis, fluorescence excitation wavelengths 330 nm, 430 nm, and 470 nm were selected, yielding a mean sensitivity of 79% and mean specificity of 79%. In the case of both sets, the sensitivity ranged from 55% to 94% and the specificity ranged from 63% to 91%.

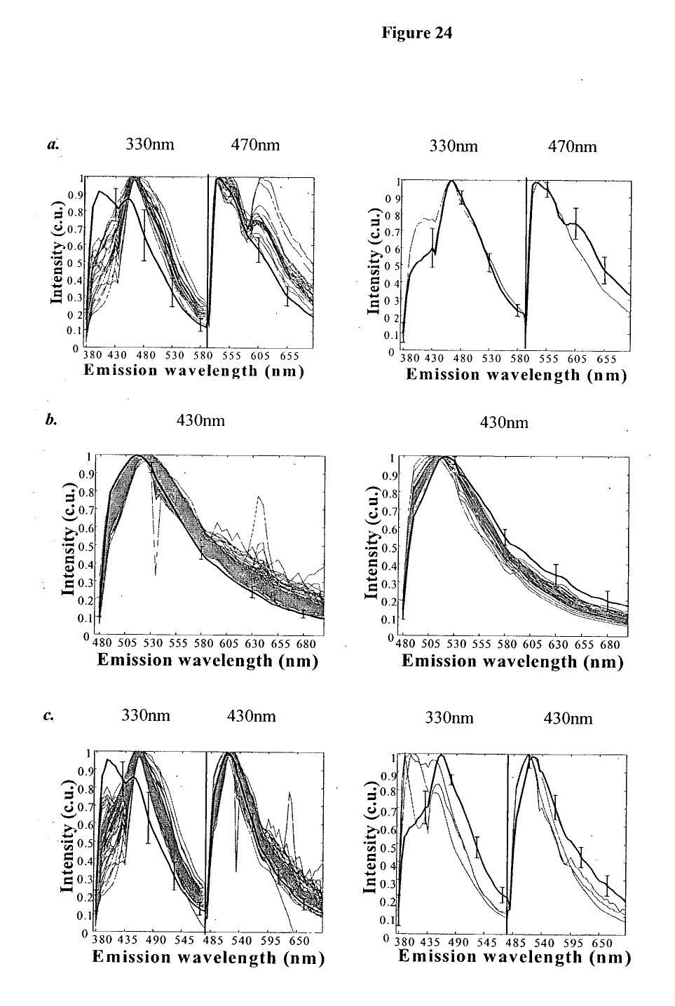

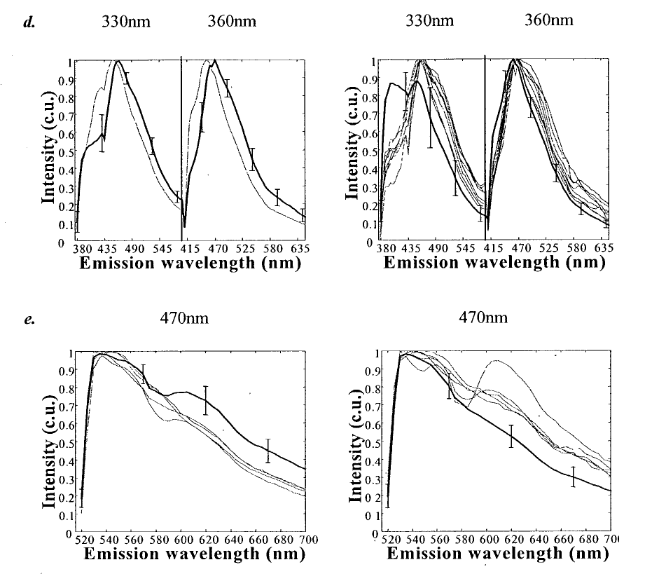

The classification of a tissue measurement sample using diagnostic category pair-wise discrimination, when using the four classification feature set identified in Table 11, is shown in FIG. 24. An average spectrum of all correctly classified tissue measurements of a diagnostic category are shown as a heavy black line average, with the individual spectra of the misclassified samples within the diagnostic category shown as gray lines. The misclassified measurements show that the important factor in classifying a measurement is peak placement/shift. In SN vs. CN, the peaks for correctly classified SN samples lie below 450 nm emission, whereas those for correctly classified CN samples lie above 450 nm emission. Although for differing emission wavelengths, the same observation can be made for nearly all the diagnostic category discrimination pairings. In CN vs. HGSIL, the slope of the spectra above 530 nm seems to be important in diagnostic category discrimination.

Discussion

In the study of the diagnostic potential of combined fluorescence and reflectance spectroscopy, cervical in vivo measurements at four distinct source-detector separation positions and for sixteen fluorescence excitation wavelengths were obtained. Using Mahalanobis distance classification, a classification combination that contained the most diagnostically useful information was determined. Results showed the sensitivity and specificity to be high when using a single classification feature at the lowest level of eigenvector significance considered. The addition of a second classification feature did maximize the sesitivity and specificity, however, there was no noticeable increase when data from higher ESLs are included. Furthermore, of the fluorescence excitation wavelengths considered, 330-360 nm and 460-470 nm appear most frequently and of the source-detector separations considered, the smaller separations of positions 0 and 1 appear most frequently in the classification feature combinations that performed best. However, the additional fluorescence excitation wavelength of 430 nm is included in nearly all the optimal classification feature sets as necessary for best discriminating squamous normal tissue from both LGSIL and HGSIL—while the source-detector separations of positions 0 and 1 did not appear in any of the optimal classification feature sets, where they were expected for use in the best discrimination of columnar normal tissue from HGSIL.

Results show that in either of the overall optimized three or four feature sets considered (TABLE ??????), both consisting of fluorescence features alone, HGSIL could be discriminated from squamous and columnar normal tissue with high sensitivity (83%, 72%) and high specificity (80%, 78%). These results indicate similar sensitivity and improved specificity for discrimination of SN vs. HGSIL found in previous studies, where sensitivities of (82%, 81%) and specificities of (67%, 73%) were obtained using analysis of reflectance spectroscopy alone. Furthermore, it was found that in both overall optimal features sets, LGSIL could be separated from columnar normal tissue with slightly higher sensitivity and specificity, (87%, 94%) in the four-feature set and (90%, 83%) in the three-feature set. However, discrimination of LGSIL and squamous normal tissue wa most difficult with a sensitivity of 55% and specificity of 63% in either overall optimal feature set. In all cases, the specificity associated with reflectance and/or fluorescence spectroscopy was significantly higher than that previously reported for colposcopy (48%), while sensitivity was somewhat lower.

### Practical Applications and Advantages of the Invention

A practical application of the invention that has value within the technological arts is for the detection of cervical precancers. This invention represents an improvement upon current technology, providing a more accurate detection of pre-cancerous cells for patients.

All the disclosed embodiments of the invention disclosed herein can be made and used without undue experimentation in light of the disclosure. The invention is not limited by theoretical statements recited herein.

### REFERENCES

Each of the reference listed are hereby incorporated by reference in their entirety.

U.S. Pat. No. 6,370,422

U.S. Pat. No. 6,258,576

U.S. Pat. No. 6,241,662

U.S. Pat. No. 6,187,289

U.S. Pat. No. 6,135,965

U.S. Pat. No. 6,095,982

U.S. Pat. No. 5,991,653

U.S. Pat. No. 5,929,985

U.S. Pat. No. 5,920,399

U.S. Pat. No. 5,842,995

U.S. Pat. No. 5,699,795

U.S. Pat. No. 5,697,373

U.S. Pat. No. 5,623,932

U.S. Pat. No. 5,612,540

U.S. Pat. No. 5,562,100

U.S. Pat. No. 5,421,339

U.S. Pat. No. 5,421,337

U.S. Pat. No. 5,419,323

U.S. Pat. No. 5,345,941

U.S. Pat. No. 5,201,318

1. A. Dellas, H. Moch, E. Schulthesis, G. Feichter, A. C. Almendral, F. Gudat, J. Torhorst, “Angiogenesis in Cervical Neoplasia: Microvessel Quantitation in Precancerous Lesions and Invasive Carcinomas with Clinicopathological Correlations,” Gyn. Onc., 67, 27-33 (1997).

2. A. Mahadevan, M. Mitchell, E. Silva, S. Thomsen, R. Richards-Kortum, “A Study of the Fluorescence Properties of Normal and Neoplastic Human Cervical Tissue,” Las. Surg. Med., 13, 647-655 (1993).

3. A. Zuluaga, U. Utzinger, A. Durkin, H. Fuchs, A. Gillenwater, R. Jacob, B. Kemp, J. Fan, R. Richards-Kortum, “Fluorescence Excitation Emission Matrices of Human Tissue: A System for In vivo Measurement and Data Analysis,” Applied Spectroscopy, submitted (1998).

4. A. J. Durkin, S. Jaikumar, R. Richards-Kortum, “Optically Dilute, Absorbing, and Turbid Phantoms for Fluorescence Spectroscopy of Homogeneous and Inhomogeneous Samples,” Applied Spectroscopy, 47(12), 2114-2121 (1993).

5. A. J. Welch, C. Gardner, R. Richards-Kortum, E. Chan, G. Criswell, J. Pfefer, S. Warren, “Propagation of Fluorescent Light,” Lasers in Surgery and Medicine, 21(2), 166-178 (1997).

6. Agrawal A, Utzinger U, Brookner C, Pitris C, Follen Mitchell M, Richards-Kortum R. Fluorescence Spectroscopy of the Cervix: Influence of Acetic Acid, Cervical Mucus, and Vaginal Medications. Lasers Surg Med, 25:237-49, 1999.

7. B. Tromberg, “Optical and Physiological Properties of Tumors,” OSA Annual Meeting, Talk (1998).

8. B. Tromberg, “Optical and Physiological Properties of Tumors,” OSA Annual Meeting, Talk (1998).

9. B. W. Pogue, G. C. Burke, J. Weaver, D. M. Harper, “Development of a Spectrally Resolved Colposcope for Early Detection of Cervical Cancer,” BME Optical Spectroscopy and Diagnostics Tech. Digest. OSA, 87-89 (1998).

10. Burke L, Modell M, Niloff J, Kobelin M, Abu-Jawdeh G, Zelenchuk A. Identification of Squamous Intraepithelial Lesions: Fluorescence of Cervical Tissue During Colposcopy. J Lower Genital Tract Disease, 3:159-62, 1999.

11. C. Brookner, U. Utzinger, G. Staerkel, R. Richards-Kortum, M. Follen Mitchell, “Cervical Fluorescence of Normal Women,” Las. Surg. Med., submitted (1998).

12. Cantor S B, Mitchell M F, Tortelero Luna G, Bratka C, Bodurka D, Richards-Kortum R. Cost-Effectiveness Analysis of Diagnosis and Management of Cervical Squamous Intraepithelial Lesion. Obstet Gynecol, 91:270-77, 1998

13. Dillon W R, Goldstein M. Multivariate Analysis: Methods and Applications. New York: John Wiley and Sons; 1984.

14. Ferris D G, Lawhead R A, Dickman E D, Holtzapple N, Miller J A, Grogan S, Bambot S, Agrawal A, Faupel M L. Multimodal Hyperspectral Imaging for the Noninvasive Diagnosis of Cervical Neoplasia. J Lower Genital Tract Disease, 5:65-72, 2001.

15. Follen Mitchell M, Cantor S B, Brookner C, Utzinger U, Schottenfeld D, Richards-Kortum R. Screening for squamous intraepithelial lesions with fluorescence spectroscopy. Obstet Gynecol, 94:889-96,1999.

16. Follen Mitchell M, Hittelman W, Hong W K, Lotan R, Shottenfeld D. The Natural History of CIN: An Argument for Intermediate Endpoint Biomarkers. Cancer Epidemiol Biomarkers Prev. 3:619-26, 1994.

17. Furlong S. Industrial Roundtable, OSA Spring Topical Meeting on Biomedical Optical Spectroscopy and Diagnostics, 1998.

18. G. Zonios, L. T. Perelman, V. Backman, R. Manoharan, M. Fitzmaurice, J. Van Dam, M. S. Feld, “Diffuse Reflectance Spectroscopy of Human Adenomatous Colon Polyps in vivo,” Appl. Opt., 38(31),6628-6637 (1999).

19. G. A. Wagnieres, W. M Star, B. C. Wilson, “In vivo Fluorescence Spectroscopy and Imaging for Oncological Applications,” Photochem. Photobiol., November 68(5), 603-32 (1998).

20. Heintzelman D L, Utzinger U, Fuchs H, Zuluaga A, Gossage K, Gillenwater A M, Jacob R, Kemp B, Richards-Kortum R R. Optimal Excitation Wavelengths for In vivo Detection of Oral Neoplasia Using Fluorescence Spectroscopy. Photochem Photobiol, 72:103-13, 2000.

21. http://ee.ogi.edu/omlc/news/feb98/polarization/index.html

22. http://www.nccc-online.org/

23. I. J. Bigio, T. R. Loree, J. Mourant, et al., “Spectroscopic Diagnosis of Bladder Cancer with Elastic Light Scattering,” Las. Surg. Med., 16 (1995).

24. J. Hung, S. Lam, J. C. LeRiche, B. Palcic, “Autofluorescence of Normal and Malignant Bronchial Tissue,” Las. Surg. Med., 11, 99-105 (1991).

25. J. A. Zuclich, T. Shimada, T. R. Loree, I. Bigio, K. Strobl, S. Nie, Lasers in the Life Sciences, 6, 39-53 (1994).

26. J. R. Mourant, A. H. Hielscher, A. A. Eick, T. M. Johnson, J. P. Freyer, “Evidence of Intrinsic Differences in the Light Scattering Properties of Tumorigenic and Nontumorigenic Cells,” Cancer, 84(6), 366-74 (1998).

27. J. R. Mourant, I. Bigio, J. Boyer, T. Johnson, J. Lacey, “Detection of GI Cancer by Elastic Scattering Spectroscopy,” J Biomed Opt., 1, 192-99 (1996).

28. J. R. Mourant, J. Boyer, A. H. Hielscher, I. J. Bigio, “Influence of the Scattering Phase Function on Light Transport Measurement in Turbid Media Performed with Small Source Detector Separations,” Optics Letters, 21(7), (1996).

29. K. Tumer, N. Ramanujam, J. Ghosh, R. Richards-Kortum, “Ensembles of Radial Basis Function Networks for Spectroscopic Detection of Cervical Precancer,” IEEE Trans BME, 45, 953-61 (1998).

30. K. T. Schomacker, J. K. Frisoli, C. Compton, et al., “Ultraviolet Laser-Induced Fluorescence of Colonic Tissue: Basic Biology and Diagnostic Potential,” Las. Surg. Med., 12, 63-78 (1992).

31. Koss, L G. The Papanicoloau test for cervical cancer detection: A triumph and a tragedy. JAMA, 261:737-43, 1989.

32. Kurman R J, Herison D E, Herbst A L, Noller K L, Schiffman M H. Interim guidelines for management of abnormal cervical cytology. JAMA, 271:1866-9,1994.

33. L. Perelman, V. Backman, M. Wallace, G. Zonios, R. Manoharan, A. Nusrat, S. Shields, M. Seiler, C. Lima, T. Hamano, I. Itzkan, J. Van Dam, J. M. Crawford, M. S. Feld, “Observation of Periodic Fine Structure in Reflectance from Biological Tissue: A New Technique for Measuring Nuclear Size Distribution,” Physical Review Letters, 80, 627-30 (1998).

34. L. Wang, S. L. Jacques, “Use of a Laser with an Oblique Angle of Incidence to Measure the Reduced Scattering Coefficient of a Turbid Media,” Appl. Opt., 34 (13), 2362, (1995).

35. L. Wang, S. L. Jacques, Med.Phys., 21, 1081 (1994).

36. Lachenbruch P A, Discriminant Analysis. Hafner Press, New York; 1975.

37. M. Coppleson, B. L. Reid, V. Skladnev, Dalrymple, “An Electronic Approach to the Detection of Precancer and Cancer of the Uterine Cervix: A Preliminary Evaluation of Polar Probe,” Int J Gynecol Cancer, 4, 79-93 (1994).

38. M. F. Mitchell, “Accuracy of Colposcopy,” Consult. Obst. Gyn., 6(1), 70-73.

39. M. G. Nichols, E. L. Hull, T. Foster, “Design and Testing of a White-Light, Steady-State Diffuse Reflectance Spectrometer for Determination of Optical Properties of Highly Scattering Systems,” Appl. Opt., 36, 93 (1997).

40. M. L. Harries, S. Lam, C. MacAulay, J. Qu, B. Palcic, “Diagnostic Imaging of the Larynx: Autofluorescence of Laryngeal Tumors Using the Helium-Cadmium Laser,” Journal of Laryngology & Otology.

41. M. T. Fahey, L. Irwig, and P. Macaskill, “Meta-analysis of pap test accuracy,” Amer. J. Epidemiol., 141(7), 680-689 (1995).

42. Mahadevan A, Mitchell M, Silva E, Thomsen S, Richards-Kortum R. A Study of the Fluorescence Properties of Normal and Neoplastic Human Cervical Tissue. Lasers Surg Med, 13:647-55, 1993.

43. N. Ramanujam, M. Follen Mitchell, A. Mahadevan, S. Thomsen, A. Malpica, T. Wright, N. Atkinson, R. Richards-Kortum, “Development of a Multivariate Statistical Algorithm to Analyze Human Cervical Tissue Fluorescence Spectra Acquired In vivo,” Las. Surg. Med., 19, 46-62 (1996).

44. N. Ramanujam, M. Follen Mitchell, A. Mahadevan, S. Thomsen, A. Malpica, T. Wright, N. Atkinson, R. Richards-Kortum, “Spectroscopic Diagnosis of Cervical Intraepithelial Neoplasia (CIN) In vivo Using Laser Induced Fluorescence Spectra at Multiple Excitation Wavelengths,” Las. Surg. Med., 19, 63-74 (1996).

45. N. Ramanujam, M. Follen Mitchell, A. Mahadevan, S. Thomsen, R. Richards-Kortum, “Fluorescence Spectroscopy as a Diagnostic Tool for Cervical Intraepithelial Neoplasia,” Gyn. Onc., 52, 31-38 (1994).

46. N. Ramanujam, M. Follen Mitchell, M. Mahadevan-Jansen, S. L. Thomsen, G. Staerkel, A. Malpica, T. Wright, N. Atkinson, R. Richards-Kortum, “Cervical Precancer Detection Using a Multivariate Statistical Algorithm Based on Laser Induced Fluorescence Spectra at Multiple Excitation Wavelengths,” Photochemistry and Photobiology, 6, 720-35 (1996).

47. N. Ramanujam, M. F. Mitchell, A. Mahadevan, S. Thomsen, E. Silva, R. Richards-Kortum, “In vivo Diagnosis of Cervical Intraepithelial Neoplasia Using 337 nm Laser Induced Fluorescence,” Proceedings of the National Academy of Sciences, 91, 10193-97 (1994).

48. R. Drezek, M. Guillaud, T. Collier, I. Boiko, A. Malpica, C. MacAulay, M. Follen, R. Richards-Kortum, “Light Scattering from Cervical Cells Throughout Neoplastic Progression: Influence of Nuclear Morphology, DNA Content, and Chromatin Texture,” Biophysical Journal, submitted (2001).

49. R. J. Kurman (ed.), Blaustein's Pathology of the Female Genital Tract, Ed. 3, New York: Springer-Verlag, 1987.

50. R. Richards-Kortum, “Role of Laser Induced Fluorescence Spectroscopy in Diagnostic Medicine,” Chapter 21 in Optical-Thermal Responses of Laser Irradiated Tissue, A. J. Welch and M. Van Gemert, Eds., Plenum Press, New York (1994).

51. R. Richards-Kortum, R. P Rava, R. E. Petras, M. Fitzmaurice, M. Sivak, M. S. Feld, “Spectroscopic Diagnosis of Colonic Dysplasia,” Photochem. Photobiol., 53(6), 777-786 (1991).

52. R. A. Zangaro, L. Silveira, R. Manoharan, G. Zonios, I. Itzkan, R. Dasari, J. Van Dam, M. S. Feld, “Rapid Multiexcitation Fluorescence Spectroscopy System for in vivo Tissue Diagnosis,” Applied Optics, September, 35(25), 5211-5219 (1996).

53. R. J. Kurman, D. E. Henson, A. L. Herbst, K. L. Noller, M. H. Schiffman, “Interim Guidelines for Management of Abnormal Cervical Cytology,” JAMA, 271, 11866-1869 (1994).

54. R. J. Nordstrom, L. Burke, J. M. Niloff, J. F. Myrtle, “Identification of Cervical Intraepithelial Neoplasia (CIN) Using UV-Excited Fluorescence and Diffuse-Reflectance Tissue Spectroscopy,” Las. Surg. Med., 29(2), 118-27 (2001).

55. R. M. Cothren, M. V. Sivak, J. Van Dam, et al., “Detection of Dysplasia at Colonoscopy Using Laser-Induced Fluorescence: A Blinded Study,” Gastrointest. Endosc., in press (1996).

56. R. R. Alfano, G. C. Tang, A. Pradham, W. Lam, D. S. J. Choy, E. Opher, “Fluorescence Spectra from Cancerous and Normal Human Breast and Lung Tissues,” IEEE J. Quant. Electron, QE, 1806-1811 (1987).

57. Ramanujam N, Mitchell M F, Mahadevan-Jansen A, Thomsen S L, Staerkel G, Malpica A, Wright T, Atkinson N, Richards-Kortum R. Cervical Pre-Cancer Detection Using a Multivariate Statistical Algorithm Based on Laser Induced Fluorescence Spectra at Multiple Excitation Wavelengths. Photochem Photobiol, 6:720-35, 1996.

58. Ramanujam N, Follen Mitchell M, Mahadevan A, Thomsen S, Malpica A, Wright T, Atkinson N, Richards-Kortum R. Development of a Multivariate Statistical Algorithm to Analyze Human Cervical Tissue Fluorescence Spectra Acquired In vivo. Lasers Surg Med, 19:46-62, 1996.

59. Ramanujam N, Mitchell M F, Mahadevan A, Thomsen S, Malpica A, Wright T, Atkinson N, Richards-Kortum R. Spectroscopic Diagnosis of Cervical Intraepithelial Neoplasia (CIN) In vivo Using Laser Induced Fluorescence Spectra at Multiple Excitation Wavelengths. Lasers Surg Med, 19:63-74, 1996.

60. Ramanujam N, Mitchell M F, Mahadevan A, Thomsen S, Malpica A, Wright T C, Atkinson N, Richards-Kortum R R. In vivo diagnosis of cervical intraepithelial neoplasia using 337-nm-excited laser-induced fluorescence. Proc Natl Acad Sci USA, 91:10193-97, 1994.

61. S. Lam, C. MacAulay, B. Palcic, “Detection and Localization of Early Lung Cancer by Imaging Techniques,” Chest, 103(1 Suppl), 12s⁻¹⁴S (1993).

62. S. Lam, D. MacAulay, J. Hung, J. LeRiche, A. E. Profio, B. Palcic, “Detection of Dysplasia and Carcinoma In Situ with a Lung Imaging Fluorescence Endoscope Device,” Journal of Thoracic & Cardiovascular Surgery, 105(6), 1035-40 (1993).

63. S. Lam, T. Kennedy, M. Unger, Y. E. Miller, D. Gelmont, V. Rusch, B: Gipe, D. Howard, J. C. LeRiche, A. Coldman, A. F. Gazdar, “Localization of Bronchial Intraepithelial Neoplastic Lesions by Fluorescence Bronchoscopy,” Chest, March 113(3), 696-702 (1998).

64. S. B. Cantor, M. Mitchell M D, G. Tortolero-Luna, C. S. Bratka, D. Bodurka, R. Richards-Kortum, “Cost-Effectiveness Analysis of the Diagnosis and Management of Cervical Intraepithelial Neoplasia,” Obst. Gyn., 91, 270-7 (1998).

65. S. K. Chang, M. Follen, A. Malpica, U. Utzinger, G. Staerkel, D. Cox, E. N. Atkinson, C. MacAulay, R. Richards-Kortum. “Optimal Excitation Wavelengths for Detection of Cervical Neoplasia,” IEEE Trans BME, submitted (2001).

66. S. P. Lin, L. Wang, S. L. Jacques, F. K. Tittel, “Measure of Tissue Optical Properties by the use of Oblique-Incidence Optical Fiber Reflectometry,” Appl. Opt., 36, 136 (1997).

67. Shomacker K, Frisoli J, Compton C, Flotte T, Richter J M, Nishioka N, Deutsch T. UV LIF of Colonic Tissue: Basic Biology and Diagnostic Potential. Lasers Surg Med, 12:63-78, 1992.

68. T. Vo-Dinh, M. Panjehpour, B. F. Overholt, C. Farris, F. P. Buckley, R. Sneed, “In vivo Cancer Diagnosis of the Esophagus Using Differential Normalized Fluorescence (DNF) Indices,” Las Surg. Med., 16, 41-47 (1995).

69. T. C. Wright, R. J. Kurman, A. Ferenczy, “Cervical Intraepithelial Neoplasia,” in Pathology of the Female Genital Tract, A. Blaustein, Eds., Springer-Verlag, New York, (1994).

70. T. J. Farell, M. S. Patterson, B. C. Wilson, “A Diffusion Theory Model of Spatially Resolved, Steady-State Diffuse Reflectance for the Non-invasive Determination of Tissue Optical Properties,” Med. Phy., 19, 879-888 (1992).

71. Trujillo E V, Sandison D R, Utzinger U, Ramanujam N, Follen Mitchell M, Richards-Kortum R R. A Method to Determine Tissue Fluorescence Efficiency in vivo and Predict Signal to Noise Ratio for Spectrometers. Applied Spectroscopy, 52:943-51, 1998.

72. U. Utzinger, V. Trujillo, E. N. Atkinson, M. F. Mitchell, S. B. Cantor, R. Richards-Kortum, “Performance Estimation of Diagnostic Tests for Cervical Precancer Based on Fluorescence Spectroscopy: Effects of Tissue Type, Sample Size, Population and Signal to Noise Ratio,” IEEE Transactions in Biomedical Engineering, submitted (1998).

73. Utzinger U, Brewer M, Silva E, Gershenson D, Bast R C, Follen M, et al. Reflectance Spectroscopy for In vivo Characterization of Ovarian Tissue. Lasers Surg Med, 28:56-66, 2001.

74. V. P. Wallace, J. C. Bamber, D. C. Crawford, R. J. Ott, P. S. Mortimer, “Classification of Reflectance Spectra from Pigmented Skin Lesions, a Comparison of Multivariate Discriminate Analysis and Artificial Neural Networks,” Phys. Med. Biol., 45, 2859-2871(2000).

75. Welch A J, Gardner C, Richards-Kortum R, Criswell G, Chan E, Pfefer J, Warren S. Propagation of Fluorescent Light. Lasers Surg Med, 21:166-78, 1997.

76. Y. Mirabal, S. K. Chang, E. N. Atkinson, A. Malpica, M. Follen, R. Richards-Kortum, “Reflectance Spectroscopy for In vivo Detection of Cervical Precancer,” Journal of Biomedical Optics, in press.

77. Zangaro R A, Silveira L, Manoharan R, Zonios G, Itzkan I, Dasari R, Van Dam J, Feld M S. Applied Optics, 35:5211-9, 1997.

78. Zuclich J A, Shimada T, Loree T R, Bigio I, Strobl K, Nie. Lasers in the Life Sciences, 6:39-53, 1994.

79. Zuluaga A, Utzinger U, Durkin A, Fuchs H, Gillenwater A, Jacob R, Kemp B, Fan J, Richards-Kortum R. Fluorescence Excitation Emission Matrices of Human Tissue: A System for in vivo Measurement and Data Analysis. Applied Spectroscopy, 53:302-11, 1998.

What is claimed is:

1. A method of detecting tissue abnormality in a tissue sample in vivo comprising:
providing a tissue sample;
sequentially illuminating the tissue sample in vivo with excitation light from a fiber optic probe;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation; and
determining from the set of reflectance spectra whether the tissue sample is normal or abnormal.

2. The method of claim 1, wherein the calculating step comprises pre-processing the set of reflectance spectra to reduce patient-to-patient variation.

3. The method of claim 1, wherein the calculating step comprises conducting principal component analysis of the reflectance spectra.

4. The method of claim 1, wherein the calculating step comprises selecting and classifying the tissue sample using Mahalanobis distance.

5. The method of claim 4, wherein the calculating step further comprises cross-validating results from selecting and classifying the tissue sample using Mahalanobis distance.

6. A method of detecting tissue abnormality in a tissue sample in vivo comprising:
providing a tissue sample;
sequentially illuminating the tissue sample in vivo with a first and second electromagnetic wavelength, the first electromagnetic wavelength being selected from the range 330-350 nm and the second electromagnetic wavelength being selected from the range 370-450 nm;
detecting the set of fluorescence intensity spectra emitted from the tissue sample as a result of illumination; and
determining from the set of fluorescence intensity spectra whether the tissue sample is normal or abnormal.

7. The method of claim 6, wherein the calculating step comprises truncating the set of fluorescence intensity spectra at 700 nm.

8. The method of claim 6, wherein the calculating step comprises pre-processing the set of fluorescence intensity spectra to reduce patient-to-patient variations.

9. The method of claim 6, wherein the calculating step comprises conducting principal component analysis of the fluorescence spectra.

10. The method of claim 6, wherein the calculating step comprises selecting and classifying the tissue sample using Mahalanobis distance.

11. The method of claim 10, wherein the calculating step further comprises cross-validating results from selecting and classifying the tissue sample using Mahalanobis distance.

12. A method of detecting tissue abnormality in a tissue sample in vivo comprising:
providing a tissue sample;
sequentially illuminating the tissue sample in vivo with a first and second electromagnetic wavelength, the first electromagnetic wavelength being selected from the range 330-340 nm and the second electromagnetic wavelength being selected from the range 410-420 nm;
detecting the set of fluorescence intensity spectra emitted from the tissue sample as a result of illumination; and
de from the set of fluorescence intensity spectra whether the tissue sample is normal or abnormal.

13. The method of claim 12, wherein the calculating step comprises truncating the set of fluorescence intensity spectra at 700 nm.

14. The method of claim 12, wherein the calculating step comprises pre-processing the set of fluorescence intensity spectra to reduce patient-to-patient variations.

15. The method of claim 12, wherein the calculating step comprises conducting principal component analysis of the fluorescence spectra.

16. The method of claim 12, wherein the calculating step comprises selecting and classifying the tissue sample using Mahalanobis distance.

17. The method of claim 16, wherein the calculating step further comprises cross-validating results from selecting and classifying the tissue sample using Mahalanobis distance.

18. A method of detecting tissue abnormality in a tissue sample in vivo comprising:
providing a tissue sample;
sequentially illuminating the tissue sample in vivo with a first and second electromagnetic wavelenth, the first electromagnetic wavelength being selected from the range 330-350 nm and the second electromagnetic wavelength being selected from the range 400-450 nm;
detecting the set of fluorescence intensity spectra emitted from the tissue sample as a result of illumination; and
determining from the set of fluorescence intensity spectra whether the tissue sample is normal or abnormal.

19. The method of claim 18, wherein the calculating step comprises truncating the set of fluorescence intensity spectra at 700 nm.

20. The method of claim 18, wherein the calculating step comprises pre-processing the set of fluorescence intensity spectra to reduce patient-to-patient variations.

21. The method of claim 18, wherein the calculating step comprises conducting principal component analysis of the fluorescence spectra.

22. The method of claim 18, wherein the calculating step comprises selecting and classifying the tissue sample using Mahalanobis distance.

23. The method of claim 22, wherein the calculating step further comprises cross-validating results from selecting and classifying the tissue sample using Mahalanobis distance.

24. A method of detecting tissue abnormality in a tissue sample in vivo comprising:
providing a tissue sample;
sequentially illuminating the tissue sample in vivo with a single electromagnetic wavelength, the single electromagnetic wavelength being selected from the range 370-400 nm;
detecting the set of fluorescence intensity spectra emitted from the tissue sample as a result of illumination; and
determining from the set of fluorescence intensity spectra whether the tissue sample is normal or abnormal.

25. The method of claim 24, wherein the calculating step comprises truncating the set of fluorescence intensity spectra at 700 nm.

26. The method of claim 24, wherein the calculating step comprises pre-processing the set of fluorescence intensity spectra to reduce patient-to-patient variations.

27. The method of claim 24, wherein the calculating step comprises conducting principal component analysis of the fluorescence spectra.

28. The method of claim 24, wherein the calculating step comprises selecting and classifying the tissue sample using Mahalanobis distance.

29. The method of claim 28, wherein the calculating step further comprises cross-validating results from selecting and classifying the tissue sample using Mahalanobis distance.

30. A method of detecting tissue abnormality in a tissue sample in vivo comprising:
providing a tissue sample;
sequentially illuminating the tissue sample in vivo with a first, second, and third electromagnetic wavelength, the first electromagnetic wavelength being selected from the range 330-340 nm, the second electromagnetic wavelength being selected from the range 350-380 nm, and the third electromagnetic wavelength being selected from the range 400-450 nm;
detecting the set of fluorescence intensity spectra emitted from the tissue sample as a result of illumination; and
determining from the set of fluorescence intensity spectra whether the tissue sample is normal or abnormal.

31. The method of claim 30, wherein the calculating step comprises truncating the set of fluorescence intensity spectra at 700 nm.

32. The method of claim 30, wherein the calculating step comprises pre-processing the set of fluorescence intensity spectra to reduce patient-to-patient variations.

33. The method of claim 30, wherein the calculating step comprises conducting principal component analysis of the fluorescence spectra.

34. The method of claim 30, wherein the calculating step comprises selecting and classifying the tissue sample using Mahalanobis distance.

35. The method of claim 34, wherein the calculating step further comprises cross-validating results from selecting and classifying the tissue sample using Mahalanobis distance.

36. A method of detecting tissue abnormality in a tissue sample in vivo comprising:
providing a tissue sample;
sequentially illuminating the tissue sample in vivo with an excitation light and a first, second, and third electromagnetic wavelength, the first electromagnetic wavelength being selected from the range 330-360 nm, the second electromagnetic wavelength being selected from the range 420-430 nm, and the third electromagnetic wavelength being selected from the range 460-470 nm;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation;
detecting the set of fluorescence intensity spectra emitted from the tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

37. The method of claim 36, wherein the at least one source-detector separation is selected from the group consisting of 250 μm separation, 1.1 mm separation, 2.1 mm separation, and 3.0 mm separation.

38. The method of claim 36, wherein the calculating step comprises truncating the set of fluorescence intensity spectra at 700 nm.

39. The method of claim 36, wherein the calculating step comprises pre-processing the set of fluorescence intensity spectra and the set of reflectance spectra to reduce patient-to-patient variations.

40. The method of claim 36, wherein the calculating step comprises conducting principal component analysis of the set of fluorescence intensity spectra and the set of reflectance spectra.

41. The method of claim 36, wherein the calculating step comprises selecting and classifying the tissue sample using Mahalanobis distance.

42. The method of claim 41, wherein the calculating step further comprises cross-validating results from selecting and classifying the tissue sample using Mahalanobis distance.

43. A method of detecting tissue abnormality in a tissue sample in vivo comprising:
providing a tissue sample;
sequentially illuminating the tissue sample in vivo with an excitation light and a first and second electromagnetic wavelength, the first electromagnetic wavelength being selected from the range 330-360 nm, and the second electromagnetic wavelength being 460 nm;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation;
detecting the set of fluorescence intensity spectra emitted from the tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

44. The method of claim 43, wherein the at least one source-detector separation is selected from the group consisting of 250 μm separation, 1.1 mm separation, 2.1 mm separation, and 3.0 mm separation.

45. The method of claim 43, wherein the calculating step comprises truncating the set of fluorescence intensity spectra at 700 nm.

46. The method of claim 43, wherein the calculating step comprises pre-processing the set of fluorescence intensity spectra and the set of reflectance spectra to reduce patient-to-patient variations.

47. The method of claim 43, wherein the calculating step comprises conducting principal component analysis of the fluorescence intensity spectra and the set of reflectance spectra.

48. The method of claim 43, wherein the calculating step comprises selecting and classifying the tissue sample using Mahalanobis distance.

49. The method of claim 48, wherein the calculating step further comprises cross-validating results from selecting and classifying the tissue sample using Mahalanobis distance.

50. A method of detecting tissue abnormality in a tissue sample in vivo comprising:
providing a tissue sample;
sequentially illuminating the tissue sample in vivo with an excitation light and a first and second set of electromagnetic wavelength, the first electromagnetic wavelength being selected from the range 330-350 nm and the second electromagnetic wavelength being 470 nm;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation;
detecting the set of fluorescence intensity spectra emitted from the tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

51. The method of claim 50, wherein the at least one source-detector separation is selected from the group consisting of 250 μm separation, 1.1 mm separation, 2.1 mm separation, and 3.0 mm separation.

52. The method of claim 50, wherein the calculating step comprises truncating the set of fluorescence intensity spectra at 700 nm.

53. The method of claim 50, wherein the calculating step comprises pre-processing the set of fluorescence intensity spectra and the set of reflectance spectra to reduce patient-to-patient variations.

54. The method of claim 50, wherein the calculating step comprises conducting principal component analysis of the fluorescence intensity spectra and the set of reflectance spectra.

55. The method of claim 50, wherein the calculating step comprises selecting and classifying the tissue sample using Mahalanobis distance.

56. The method of claim 55, wherein the calculating step further comprises cross-validating results from selecting and classifying the tissue sample using Mahalanobis distance.

57. A method of detecting tissue abnormality in a tissue sample in vivo comprising:
providing a tissue sample;
sequentially illuminating the tissue sample in vivo with an excitation light and a first and second electromagnetic wavelength, the first electromagnetic wavelength being selected from the range 330-350 nm and the second electromagnetic wavelength being selected from the range 470-480 nm;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation;
detecting the set of fluorescence intensity spectra emitted from the tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

58. The method of claim 57, wherein the at least one source-detector separation is selected from the group consisting of 250 μm separation, 1.1 mm separation, 2.1 mm separation, and 3.0 mm separation.

59. The method of claim 57, wherein the calculating step comprises truncating the set of fluorescence intensity spectra at 700 nm.

60. The method of claim 57, wherein the calculating step comprises pre-processing the set of fluorescence intensity spectra and the set of reflectance spectra to reduce patient-to-patient variations.

61. The method of claim 57, wherein the calculating step comprises conducting principal component analysis of the fluorescence intensity spectra and the set of reflectance spectra.

62. The method of claim 57, wherein the calculating step comprises selecting and classifying the tissue sample using Mahalanobis distance.

63. The method of claim 62, wherein the calculating step further comprises cross-validating results from selecting and classifying the tissue sample using Mahalanobis distance.

64. A method of detecting tissue abnormality in a tissue sample in vivo comprising:
providing a tissue sample;
sequentially illuminating the tissue sample in vivo with an excitation light and a first, second, and third electromagnetic wavelength, the first electromagnetic wavelength being selected from the range 350-360 nm, the second electromagnetic wavelength being selected from the range 420-430 nm, and the third electromagnetic wavelength being 460 nm;
detecting, with the fiber optic probe, a set of reflectance spectra emitted from the tissue sample as a result of illumination with the excitation light, the fiber optic probe comprising at least one collection fiber positioned at at least one source-detector separation selected from the group consisting of 250 μm separation, 1.1 mm separation, 2.1 mm separation, and 3.0 mm separation;
detecting the set of fluorescence intensity spectra emitted from the-tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

65. The method of claim 64, wherein the at least one source-detector separation is selected from the group consisting of 250 μm separation, 1.1 mm separation, 2.1 mm separation, and 3.0 mm separation.

66. The method of claim 64, wherein the calculating step comprises truncating the set of fluorescence intensity spectra at 700 nm.

67. The method of claim 64, wherein the calculating step comprises pre-processing the set of fluorescence intensity spectra and the set of reflectance spectra to reduce patient-to-patient variations.

68. The method of claim 64, wherein the calculating step comprises conducting principal component analysis of the fluorescence intensity spectra and the set of reflectance spectra.

69. The method of claim 64, wherein the calculating step comprises selecting and classifying the tissue sample using Mahalanobis distance.

70. The method of claim 69, wherein the calculating step further comprises cross-validating results from selecting and classifying the tissue sample using Mahalanobis distance.

71. A method of detecting tissue abnormality in a tissue sample in vivo comprising:
providing a tissue sample;
sequentially illuminating the tissue sample in vivo with an excitation light and a first and second electromagnetic wavelength, the first electromagnetic wavelength being selected from the range 330-350 nm, the second electromagnetic wavelength being selected from the range 460-470 nm;
detecting, with the fiber optic probe, a set of reflectance spectra emitted from the tissue sample as a result of illumination with the excitation light, the fiber optic probe comprising at least two collection fiber positioned at at least one source-detector separation selected from the group consisting of 250 μm separation, 1.1 mm separation, 2.1 mm separation, and 3.0 mm separation;
detecting the set of fluorescence intensity spectra emitted from the tissue sample as a result of illumination; and
determining from the set of fluorescence intensity spectra or the set of reflectance spectra, or a combination of the set of fluorescence intensity spectra and the set of reflectance spectra whether the tissue sample is normal or abnormal.

72. The method of claim 71, wherein the at least one source-detector separation is selected from the group consisting of 250 μm separation, 1.1 mm separation, 2.1 mm separation, and 3.0 mm separation.

73. The method of claim 71, wherein the calculating step comprises truncating the set of fluorescence intensity spectra at 700 nm.

74. The method of claim 71, wherein the calculating step comprises pre-processing the set of fluorescence intensity spectra and the set of reflectance spectra to reduce patient-to-patient variations.

75. The method of claim 71, wherein the calculating step comprises conducting principal component analysis of the fluorescence intensity spectra and the set of reflectance spectra.

76. The method of claim 71, wherein the calculating step comprises selecting and classifying the tissue sample using Mahalanobis distance.

77. The method of claim 76, wherein the calculating step further comprises cross-validating results from selecting and classifying the tissue sample using Mahalanobis distance.
